(19) United States
(12) Patent Application Publication
(10) Pub. No.: US 2026/0263463 A1
(43) Pub. Date: Sep. 10, 2026

(54) FORMULATIONS OF DANICOPAN AND METHODS OF USE THEREOF

(71) Applicant: Alexion Pharmaceuticals, Inc., Boston, MA (US)

(72) Inventors: Jennifer CHU, Ivoryton, CT (US); Mayur DUDHEDIA, Brookfield, CT (US)

(21) Appl. No.: 19/164,701

(22) PCT Filed: Feb. 29, 2024
(86) PCT No.: PCT/US2024/017823

§ 371 Date: Sep. 12, 2025

Related U.S. Application Data
(60) Provisional application No. 63/451,894, filed on Mar. 13, 2023.

(51) Int. Cl. A61K 31/506; A61K 9/16; A61K 9/20

(52) U.S. Cl. CPC A61K 31/506; A61K 9/1617; A61K 9/1635; A61K 9/1652; A61K 9/1694; A61K 9/2009; A61K 9/2013; A61K 9/2018; A61K 9/2054

(57) ABSTRACT

This present disclosure features solid dispersion and liquid gelatin capsule formulations of danicopan that can improve the oral bioavailability and/or shelf stability of danicopan. The present disclosure also features methods for treating complement factor D mediated disorders, including paroxysmal nocturnal hemoglobinuria (PNH), e.g., PNH with clinically evident extravascular hemolysis (EVH), and geographic atrophy (GA) secondary to age-related macular degeneration (AMD) using the formulations.

### CROSS-REFERENCE TO RELATED APPLICATIONS

This application claims the benefit of U.S. Provisional Application No. 63/451,894, filed on Mar. 13, 2023, which is incorporated by reference herein in its entirety.

### FIELD OF THE DISCLOSURE

This disclosure features formulations for improving the oral bioavailability and/or the shelf stability of danicopan, and methods for treating complement factor D mediated disorders, including paroxysmal nocturnal hemoglobinuria (PNH), e.g., PNH with clinically evident extravascular hemolysis (EVH), and geographic atrophy (GA) secondary to age-related macular degeneration (AMD) using the formulations.

### BACKGROUND OF THE DISCLOSURE

The complement system is a part of the innate immune system which does not adapt to changes over the course of the host's life, but is recruited and used by the adaptive immune system. For example, it assists, or complements, the ability of antibodies and phagocytic cells to clear pathogens. This sophisticated regulatory pathway allows rapid reaction to pathogenic organisms while protecting host cells from destruction. Over thirty proteins and protein fragments make up the complement system. These proteins act through opsonization (enhancing phagocytosis of antigens), chemotaxis (attracting macrophages and neutrophils), cell lysis (rupturing membranes of foreign cells), and agglutination (clustering and binding of pathogens together).

The complement system has three pathways: classical, alternative, and lectin. Complement Factor D plays an early and central role in activation of the alternative pathway of the complement cascade. Activation of the alternative complement pathway is initiated by spontaneous hydrolysis of a thioester bond within the C3 protein to produce C3(H₂O), which associates with Factor B to form the C3(H₂O)B complex. Complement Factor D acts to cleave Factor B within the C3(H₂O)B complex to form Ba and Bb. The Bb fragment remains associated with C3(H₂O) to form the alternative pathway C3 convertase C3(H₂O)Bb. Additionally, C3b generated by any of the C3 convertases also associates with Factor B to form C3bB, which Factor D cleaves to generate the later stage alternative pathway C3 convertase C3bBb. This latter form of the alternative pathway C3 convertase may provide important downstream amplification within all three of the defined complement pathways, leading ultimately to the recruitment and assembly of additional factors in the complement cascade pathway, including the cleavage of C5 to C5a and C5b. C5b acts in the assembly of factors C6, C7, C8, and C9 into the membrane attack complex, which can destroy pathogenic cells by lysing the cell.

The dysfunction of or excessive activation of complement has been linked to certain autoimmune, inflammatory, and neurodegenerative diseases, as well as ischemia-reperfusion injury and cancer. For example, activation of the alternative pathway of the complement cascade contributes to the production of C3a and C5a, both potent anaphylatoxins, which also have roles in a number of inflammatory disorders. Therefore, in some instances, it is desirable to decrease the response of the complement pathway, including the alternative complement pathway.

Factor D is an attractive target for inhibition or regulation of the complement cascade due to its early and essential role in the alternative complement pathway, and for its potential role in signal amplification within the classical and lectin complement pathways. Inhibition of Factor D effectively interrupts the pathway and attenuates the formation of the membrane attack complex.

Given the wide variety of medical disorders that are caused by detrimental immune or inflammatory responses, it would be beneficial to provide additional advantageous compounds and formulations thereof for advantageous delivery that may increase therapeutic activity and/or stability.

### SUMMARY

The present disclosure features formulations and methods for administering danicopan. The formulations of danicopan can improve the oral bioavailability and/or shelf stability of danicopan, a proximal complement alternative pathway factor D inhibitor.

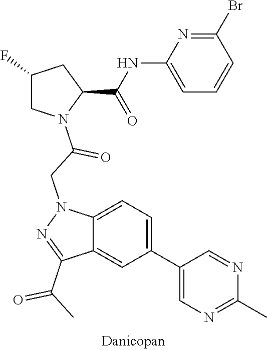 [SMILES: CC(=O)c1nn(CC(=O)N2C[C@H](F)C[C@H]2C(=O)Nc2cccc(Br)n2)c2ccc(-c3cnc(C)nc3)cc12]

The present disclosure also features uses of these formulations to inhibit AP C3 convertase formation and for treating various conditions associated with complement factor D mediated disorders, such as paroxysmal nocturnal hemoglobinuria (PNH), e.g., PNH with clinically evident extravascular hemolysis (EVH), and geographic atrophy (GA) secondary to age-related macular degeneration (AMD).

In a first aspect, the present disclosure features a solid dispersion including: (i) substantially amorphous danicopan; and (ii) a pharmaceutically acceptable polymer, wherein the (w/w) ratio of substantially amorphous danicopan to pharmaceutically acceptable polymer in the solid dispersion is from 10:1 to 1:4 (e.g., from 10:1 to 1:1; from 10:1 to 5:1; from 5:1 to 1:2; from 2:1 to 1:4; or from 1:1 to 1:4).

In certain embodiments, the percentage loading of danicopan in the solid dispersion is from 25% to 90% (w/w) (e.g., from 25% to 30%, from 25% to 40%, from 25% to 50%, from 25% to 60%, from 25% to 70%, from 25% to 80%, from 25% to 90%, from 30% to 40%, from 30% to 50%, from 30% to 60%, from 30% to 70%, from 30% to 80%, from 30% to 90%, from 40% to 50%, from 40% to 60%, from 40% to 70%, from 40% to 80%, from 40% to 90%, from 50% to 60%, from 50% to 70%, from 50% to 80%, from 50% to 90%, from 60% to 70%, from 60% to 80%, from 60% to 90%, from 70% to 80%, from 70% to 90%, from 80% to 90%, and from 80% to 90%). For example, in one embodiment, the percentage loading of danicopan in the solid dispersion is from 80% to 90%.

In certain embodiments, the (w/w) ratio of substantially amorphous danicopan to the pharmaceutically acceptable polymer in the solid dispersion is from 3:1 to 4:1. For example, in one embodiment, the (w/w) ratio of substantially amorphous danicopan to the pharmaceutically acceptable polymer is 4:1.

In certain embodiments, the solid dispersion contains at least 90% (e.g., at least 95%, 96%, 97%, 98%, 99%, 99.5%, or 99.9%, such as from 90% to 99.9%, from 90% to 99.5%, from 90% to 99%, from 90% to 98%, from 90% to 97%, from 90% to 96%, from 90% to 95%, from 95% to 99.9%, from 95% to 99.5%, from 95% to 99%, from 95% to 98%, from 95% to 97%, and from 95% to 96%) of danicopan in amorphous form, as determined by X-ray powder diffraction.

In certain embodiments, the pharmaceutically acceptable polymer in the solid dispersion includes a polymer selected from a cellulose derivative, a polyacrylate, a polyvinyl pyrrolidone, a polyvinyl acetate, a copolymer of a polyvinyl pyrrolidone and a polyvinyl acetate, and combinations thereof.

In further embodiments, the pharmaceutically acceptable polymer in the solid dispersion is a cellulose derivative including cellulose acetate having from 10% to 50% (e.g., from 10% to 15%, from 10% to 20%, from 10% to 25%, from 10% to 30%, from 10% to 35%, from 10% to 40%, from 10% to 45%, from 20% to 25%, from 20% to 30%, from 20% to 35%, from 20% to 40%, from 20% to 45%, from 20% to 50%, from 25% to 30%, from 25% to 35%, from 25% to 40%, from 25% to 45%, from 25% to 50%, from 30% to 35%, from 30% to 40%, from 30% to 45%, from 30% to 50%, from 35% to 40%, from 35% to 45%, from 35% to 50%, from 40% to 45%, or from 40% to 50%) acetyl.

In further embodiments, the pharmaceutically acceptable polymer in the solid dispersion is a cellulose derivative selected from an alkyl cellulose (e.g., methyl cellulose or ethyl cellulose), a hydroxyalkyl cellulose (e.g., hydroxymethyl cellulose; hydroxyethyl cellulose; hydroxypropyl cellulose, such as those having 11% hydroxypropyl or 8% hydroxypropyl; or hydroxybutyl cellulose), a hydroxyalkylalkyl cellulose (e.g., hydroxyethylmethyl cellulose; or hydroxypropylmethyl cellulose (HPMC) having 19-24% methoxyl/7-12% hydroxypropoxyl, 28-30% methoxyl/7-12% hydroxypropoxyl, 23% methoxyl/10% hydroxypropoxyl, 23%-29% methoxyl/8%-9% hydroxypropoxyl, 29% methoxyl/9% hydroxypropoxyl, or 23% methoxyl/6% hydroxypropoxyl), a hydroxyalkylalkyl cellulose ester (e.g., hydroxypropylmethyl cellulose phthalate (HPMCP)), a carboxyalkyl cellulose (e.g., carboxymethyl cellulose or an alkali metal salt thereof, such as a sodium salt), a carboxyalkylalkyl cellulose (e.g., carboxymethylethyl cellulose), and a carboxyalkyl cellulose ester (e.g., carboxymethyl cellulose butyrate, carboxymethyl cellulose propionate, carboxymethyl cellulose acetate butyrate, or carboxymethyl cellulose acetate propionate). In further embodiments, the cellulose derivative in the solid dispersion is cross-linked or copolymerized (e.g., with any polymer described herein). In further embodiments, the pharmaceutically acceptable polymer is a hydroxyalkylalkyl cellulose, e.g., HPMC E3.

In further embodiments, the cellulose acetate in the solid dispersion is cellulose acetate phthalate (CAP) (e.g., having 35% phthalyl/24% acetyl), methylcellulose acetate phthalate, hydroxypropylmethyl cellulose acetate, and hydroxypropylmethyl cellulose acetate succinate (HPMCAS) (e.g., having 9% acetyl/11% succinoyl, 12% acetyl/6% succinoyl, and 8% acetyl/15% succinoyl). In further embodiments, the HPMCAS polymer in the solid dispersion is selected from grade L (HPMCAS-L), grade H (HPMCAS-H), or grade M (HPMCAS-M). The HPMCAS-L polymer may consist of 5-9% acetyl, 14-18% succinoyl, 20-24% methoxyl, and 5-9% hydroxypropoxy content. The HPMCAS-M polymer may consist of 7-11% acetyl, 10-14% succinoyl, 21-25% methoxyl, and 5-9% hydroxypropoxy content. The HPMCAS-H polymer may consist of 10-14% acetyl, 4-8% succinoyl, 22-26% methoxyl, and 6-10% hydroxypropoxy content. In some embodiments, the mean particle size of the solid dispersion in the solid dispersion is 1-80 μm. In some embodiments, the mean particle size of the solid dispersion in the solid dispersion 5-75 μm.

In some embodiments, the pharmaceutically acceptable polymer in the solid dispersion is a polyacrylate selected from a polymethacrylate, a methacrylate copolymer (e.g., a methacrylic acid-methyl methacrylate copolymer having a 1:1 ratio of free carboxyl groups to ester groups and a 1:2 ratio of free carboxyl groups to ester groups, a dimethylaminoethyl methacrylate-butyl methacrylate-methyl methacrylate copolymer, and a diethylaminoethyl methacrylic acid-methyl methacrylate copolymer), and an ethacrylate copolymer (e.g., a methacrylic acid ethacrylate copolymer having a 50:50 ratio of methacrylic acid to ethacrylate). In further embodiments, the pharmaceutically acceptable polymer is a methacrylate copolymer, e.g., Eudragit L-100.

In some embodiments, the pharmaceutically acceptable polymer in the solid dispersion is a polyvinyl acetate selected from a polyvinyl pyrrolidone (e.g., povidone) having a molecular weight of more than 2,000 Da (e.g., 2,500, 9,000, 50,000, or 1,250,000 Da), a polyvinyl acetate ester (e.g., polyvinyl acetate phthalate (PVAP)), and a polyethylene glycol polyvinylacetate copolymer (e.g., polyethylene glycol-polyvinylcaprolactam-polyvinylacetate copolymer). In some embodiments, the pharmaceutically acceptable polymer is a polyvinyl acetate ester, e.g., polyvinylacetate phthalate (PVAP).

In other embodiments, the pharmaceutically acceptable polymer in the solid dispersion is a copolymer of a polyvinyl pyrrolidone and a polyvinyl acetate and the copolymer has from 10:90 to 70:30 ratio (e.g., 20:80, 30:70, 40:60, 50:50, and 60:40) of N-vinyl-2-pyrrolidone to vinyl acetate.

In another aspect, the present disclosure features a pharmaceutical composition in a capsule or tablet for oral administration that includes any one of the solid dispersions disclosed herein. The pharmaceutical composition may contain from 20 mg to 900 mg (e.g., from 20 mg to 30 mg, from 20 mg to 40 mg, from 20 mg to 50 mg, from 20 mg to 75 mg, from 20 mg to 100 mg, from 20 mg to 125 mg, from 20 mg to 150 mg, from 20 mg to 175 mg, from 20 mg to 200 mg, from 20 mg to 225 mg, from 30 mg to 40 mg, from 30 mg to 50 mg, from 30 mg to 75 mg, from 30 mg to 100 mg, from 30 mg to 125 mg, from 30 mg to 150 mg, from 30 mg to 175 mg, from 30 mg to 200 mg, from 30 mg to 225 mg, from 30 mg to 250 mg, from 40 mg to 50 mg, from 40 mg to 75 mg, from 40 mg to 100 mg, from 40 mg to 125 mg, from 40 mg to 150 mg, from 40 mg to 175 mg, from 40 mg to 200 mg, from 40 mg to 225 mg, from 40 mg to 250 mg, from 50 mg to 75 mg, from 50 mg to 100 mg, from 50 mg to 125 mg, from 50 mg to 150 mg, from 50 mg to 175 mg, from 50 mg to 200 mg, from 50 mg to 225 mg, from 50 mg to 250 mg, from 60 mg to 75 mg, from 60 mg to 100 mg, from 60 mg to 125 mg, from 60 mg to 150 mg, from 60 mg to 175 mg, from 60 mg to 200 mg, from 60 mg to 225 mg, from 60 mg to 250 mg, from 70 mg to 75 mg, from 70 mg to 100 mg, from 70 mg to 125 mg, from 70 mg to 150 mg, from 70 mg to 175 mg, from 70 mg to 200 mg, from 70 mg to 225 mg, from 70 mg to 250 mg, from 80 mg to 100 mg, from 80 mg to 125 mg, from 80 mg to 150 mg, from 80 mg to 175 mg, from 80 mg to 200 mg, from 80 mg to 225 mg, from 80 mg to 250 mg, from 90 mg to 100 mg, from 90 mg to 125 mg, from 90 mg to 150 mg, from 90 mg to 175 mg, from 90 mg to 200 mg, from 90 mg to 225 mg, from 90 mg to 250 mg, from 100 mg to 125 mg, from 100 mg to 150 mg, from 100 mg to 175 mg, from 100 mg to 200 mg, from 100 mg to 225 mg, from 100 mg to 250 mg, 200 mg to 225 mg, from 200 mg to 250 mg, from 200 mg to 275 mg, from 200 mg to 300 mg, from 200 mg to 325 mg, and from 200 mg to 350 mg, 300 mg to 325 mg, from 300 mg to 350 mg, from 300 mg to 375 mg, from 300 mg to 400 mg, from 300 mg to 425 mg, from 300 mg to 450 mg, from 400 mg to 425 mg, from 400 mg to 450 mg, from 400 mg to 475 mg, from 400 mg to 500 mg, from 400 mg to 525 mg, from 400 mg to 550 mg, from 500 mg to 525 mg, from 500 mg to 550 mg, from 500 mg to 575 mg, from 500 mg to 600 mg, from 500 mg to 625 mg, from 500 mg to 650 mg, from 600 mg to 625 mg, from 600 mg to 650 mg, from 600 mg to 675 mg, from 600 mg to 700 mg, from 600 mg to 725 mg, from 600 mg to 750 mg, from 700 mg to 725 mg, from 700 mg to 750 mg, from 700 mg to 775 mg, from 700 mg to 800 mg, from 700 mg to 825 mg, from 700 mg to 850 mg, from 800 mg to 825 mg, from 800 mg to 850 mg, from 800 mg to 875 mg, and from 800 mg to 900 mg) of substantially amorphous danicopan. In some embodiments, the pharmaceutical composition contains 100 mg substantially amorphous danicopan. In some embodiments, the pharmaceutical composition contains 50 mg substantially amorphous danicopan.

In some embodiments, the pharmaceutical composition further includes a plasticizer. Exemplary plasticizers include, but are not limited to, a polyalkylene oxide (e.g., polyethylene glycols, such as PEG 300, PEG 400, PEG 4000, and PEG 8000, and polypropylene glycols), a copolymer of ethylene oxide and propylene oxide (e.g., ethoxylated propoxylated block copolymers having the formula H(OCH₂CH₂)ₐ(OCHCH₃CH₂)_{b}(OCH₂CH₂)ₐOH, in which a is between 10 and 150 and b is between 10 and 100 (e.g., a is 12 and b is 20, a is 38 and b is 29, a is 80 and b is 27, a is 64 and b is 37, a is 141 and b is 44, a is 49 and b is 57, and a is 101 and b is 56), and a polyethoxylated glyceryl ester (e.g., polyoxyl 35 castor oil and polyoxyl 40 castor oil having 40-45 moles of ethylene oxide).

In some embodiments, the pharmaceutical composition further includes pharmaceutically acceptable binders, such as starch, gelatin, natural sugars such as glucose or beta-lactose, corn sweeteners, natural and synthetic gums such as acacia, tragacanth, or sodium alginate, carboxymethylcellulose, polyethylene glycol, and waxes.

In some embodiments, the pharmaceutical composition further includes a filler (e.g., sucrose; sorbitol; mannitol; microcrystalline cellulose; a starch, e.g., potato starch; kaolin, calcium carbonate; sodium chloride; lactose (e.g., Granulose 200 or Granulac 200); calcium phosphate; calcium sulfate; and sodium phosphate).

In some embodiments, the pharmaceutical composition further includes a pharmaceutically acceptable carrier. Exemplary pharmaceutically acceptable carriers include, but are not limited to sugars, starches, celluloses, powdered tragacanth, malt, gelatin, talc, and vegetable oils.

In some embodiments, the pharmaceutical composition further includes a pharmaceutically acceptable excipient (e.g., lactose monohydrate, such as granulated alpha-lactose monohydrate (e.g., Tablettose 80); fumed silica, such as Aerosil; magnesium stearate; croscarmellose sodium; colloidal silicon dioxide; and microcrystalline cellulose, such as Avicel PH101 and Avicel PH102); a colorant; a flavoring agent; a plasticizer; a humectant; a buffering agent; an antioxidant; a coating or a film former; a compression aid; an emollient; an emulsifier; a fragrance; a preservative; a printing ink; a sorbent; a suspending or dispersing agent; a sweetener; and waters of hydration. In some embodiments, the pharmaceutical composition further includes croscarmellose sodium. In some embodiments, the pharmaceutical composition further includes magnesium stearate. In some embodiments, the pharmaceutical composition further includes fumed silica, e.g., Aerosil. In some embodiments, the pharmaceutical composition further includes granulated alpha-lactose monohydrate, e.g., Tablettose 80. In some embodiments, the pharmaceutical composition further includes microcrystalline cellulose. In some embodiments, the microcrystalline cellulose is Avicel PH101. In some embodiments, the microcrystalline cellulose is Avicel PH102. In some embodiments, the pharmaceutical composition further includes croscarmellose sodium, magnesium stearate, fumed silica, lactose monohydrate, microcrystalline cellulose, microcrystalline cellulose, lactose, sodium lauryl sulfate, and colloidal silicon dioxide.

In some embodiments, the pharmaceutical composition further includes a surfactant. Exemplary surfactants include those selected from a polyethoxylated ester of one or more fatty acids (e.g., sodium lauryl sulfate, sodium laureth sulfate), a polyethoxylated alkyl ether, a polyethoxylated glyceryl ester, a polyoxyethylene glyceryl ester of one or more fatty acids, a sorbitan ester, a polyethoxylated sorbitan ester, a polyethoxylated vitamin analog (e.g., a TPGS, e.g., D-alpha-tocopheryl PEG 1000 succinate), and an ethoxylated propoxylated block copolymer. In some embodiments, the pharmaceutical composition further includes sodium lauryl sulphate.

In other embodiments, the solid dispersion in the pharmaceutical composition is formed by spray drying a liquid mixture including danicopan, the pharmaceutically acceptable polymer (e.g., any one of the pharmaceutically acceptable polymers disclosed herein), a pharmaceutically acceptable solvent (e.g., acetone, dichloromethane, methanol, and/or water), and pharmaceutically acceptable surfactant (e.g., sodium lauryl sulfate). In some embodiments, the polymer is HPMCAS-H, HPMCAS-L, HPMCAS-M, polyvinyl pyrrolidone, polyvinyl pyrrolidone/vinyl acetate, HPMC E3, or Eudragit L-100.

In other embodiments, the pharmaceutical composition is formulated as a capsule (e.g., a hard hydroxy propyl methylcellulose capsule, and hard gelatin capsule) wherein the capsule includes a powder including the solid dispersion. In some embodiments, the pharmaceutical composition is formulated as a tablet.

In further embodiments, the present disclosure features a pharmaceutical composition when tested in accordance with the dissolution method defined herein employing 0.1 N hydrochloric acid in water at 37° C. with 50 rpm stirring, dissolves at least 50% (w/w) (e.g., 50-60%, 55-65%, 60-70%, or 65-90% (w/w)) of the pharmaceutical composition within the first 8-90 minutes of the test.

In further embodiments, the present disclosure features a pharmaceutical composition when tested in accordance with the supersaturation profiling method defined herein employing simulated gastric fluid (SGF) or simulated intestinal fluid (SIF) at 37° C., releases 0.027 or 0.8 mg/mL of the pharmaceutical composition in SIF or SGF, respectively, within the first four hours of the test.

In another aspect, the present disclosure features a pharmaceutical composition including danicopan dissolved in a glyceride fatty acid ester, a pharmaceutically acceptable organic solvent, and a D-α-tocopherol polyethylene glycol succinate (TPGS) compound contained in a liquid soft gelatin capsule for oral administration.

In further embodiments, the pharmaceutical composition includes a ratio (w/w) of glyceride fatty acid ester, danicopan, pharmaceutically acceptable organic solvent, and TPGS of 16:2:1:1 in a liquid soft gelatin capsule.

In another aspect, the present disclosure features a pharmaceutical composition including danicopan dissolved in Capmul MCM, propylene glycol, and D-α-tocopheryl PEG 1000 succinate contained in a liquid soft gelatin capsule. The pharmaceutical composition may contain 50 mg to 400 mg (e.g., from 50 mg to 75 mg, from 50 mg to 100 mg, from 50 mg to 125 mg, from 50 mg to 150 mg, from 50 mg to 175 mg, from 50 mg to 200 mg, from 50 mg to 225 mg, from 50 mg to 250 mg, from 60 mg to 75 mg, from 60 mg to 100 mg, from 60 mg to 125 mg, from 60 mg to 150 mg, from 60 mg to 175 mg, from 60 mg to 200 mg, from 60 mg to 225 mg, from 60 mg to 250 mg, from 70 mg to 75 mg, from 70 mg to 100 mg, from 70 mg to 125 mg, from 70 mg to 150 mg, from 70 mg to 175 mg, from 70 mg to 200 mg, from 70 mg to 225 mg, from 70 mg to 250 mg, from 80 mg to 100 mg, from 80 mg to 125 mg, from 80 mg to 150 mg, from 80 mg to 175 mg, from 80 mg to 200 mg, from 80 mg to 225 mg, from 80 mg to 250 mg, from 90 mg to 100 mg, from 90 mg to 125 mg, from 90 mg to 150 mg, from 90 mg to 175 mg, from 90 mg to 200 mg, from 90 mg to 225 mg, from 90 mg to 250 mg, from 100 mg to 125 mg, from 100 mg to 150 mg, from 100 mg to 175 mg, from 100 mg to 200 mg, from 100 mg to 225 mg, from 100 mg to 250 mg, 200 mg to 225 mg, from 200 mg to 250 mg, from 200 mg to 275 mg, from 200 mg to 300 mg, from 200 mg to 325 mg, and from 200 mg to 350 mg, 300 mg to 325 mg, from 300 mg to 350 mg, from 300 mg to 375 mg, from 300 mg to 400 mg) of danicopan.

In another aspect, the present disclosure features a method of preparing any one of the solid dispersions disclosed herein. The method includes: (i) dissolving danicopan and a pharmaceutically acceptable polymer (e.g., any one of the pharmaceutically acceptable polymers disclosed herein) in an organic solvent (e.g., one or more of acetone, methanol, ethanol, and dichloromethane) to form a solution; and (ii) spray drying said solution to form the solid dispersion. In further embodiments, the organic solvent is an organic solvent with a boiling point of less than 80° C.

In other embodiments, the present disclosure features a method to treat a condition modulated by complement factor D in a subject in need of such treatment. The method includes orally administering to the subject an effective amount of the pharmaceutical composition.

In further embodiments, the present disclosure features a method where the pharmaceutical composition is orally administered once, twice, or three times per day.

In further embodiments, the present disclosure features a method where the condition to be treated is paroxysmal nocturnal hemoglobinuria (PNH) and PNH with clinically evident extravascular hemolysis (EVH). In further embodiments, the method includes a capsule or tablet that is orally administered TID with 100-200 mg of danicopan in a pharmaceutical composition.

In further embodiments, the present disclosure features a method where the condition to be treated is geographic atrophy (GA) secondary to age-related macular degeneration (AMD). In further embodiments, the method includes a capsule or tablet includes 20-900 mg of danicopan and is orally administered three times daily.

### Definitions

By “pharmaceutically acceptable polymer” is meant a polymer suitable for pharmaceutical formulation and capable of forming a solid dispersion.

As used herein, the term “substantially amorphous,” in reference to danicopan, refers to a sample of danicopan in which in which less than 20% (w/w) (e.g., less than 15%, 12%, 10%, 8%, 5%, 3%, 2%, or 1% (w/w) is present in a crystalline form, such as between 0.01% and 20%, 0.01% and 15%, 0.01% and 12%, 0.01% and 10%, 0.01% and 8%, 0.01% and 5%, 0.01% and 3%, and 0.01% and 1% (w/w)) of the danicopan is present in a crystalline form is in crystalline form, e.g., as determined using the X-ray powder diffraction method described in Example 3. In some embodiments, a substantially amorphous solid dosage form is a solid dosage form in which less than 2% (w/w) of danicopan is present in a crystalline form. Known crystalline forms of danicopan are described in WO2020/051538 and CN113801189A, the contents of which are incorporated herein by reference in their entirety. In some embodiments, the absence of crystalline danicopan is determined using differential scanning calorimetry (DSC), e.g., as described in WO2020/051538, in which the absence of a melting endotherm, e.g., at 155.3° C., indicates the absence of crystalline danicopan. As used herein, the term “solid dispersion” solid formulations including (i) substantially amorphous danicopan dispersed in a polymer. In certain dosage forms of the present disclosure, the solid dispersion is a form having homogenously dispersed danicopan throughout the polymer in a manner that results in a single glass transition temperature T_{g}.

As used herein, “bioavailability” refers to the fraction of drug absorbed following administration to a subject or patient under a fasted state.

By “coefficient of variation” is the arithmetic standard deviation divided by the arithmetic mean for a particular pharmacokinetic parameter, where the data is obtained from a pharmacokinetic study involving 10, 12, or more subjects or patients.

By “mean” is the arithmetic mean for a particular pharmacokinetic parameter, where the data is obtained from a pharmacokinetic study involving 10, 12, or more subjects or patients.

By “Cₘₐₓ” is meant the mean peak concentration of a drug achieved in plasma after dosing.

By “Tₘₐₓ” is meant the mean time after oral administration of a drug when the maximum plasma concentration of the drug or Cₘₐₓ is reached.

By “AUC_{∞}”, “AUC_{0-∞},” or “Area Under the Curve_{∞}” is meant the mean integrated area under the curve for the plasma concentration of a drug, versus time from t=0 to t=∞ following dosing.

By “TPGS (tocopherol polyethylene glycol succinate)” is meant a compound or mixture of compounds containing one or more vitamin E moieties (e.g., a tocopherol, tocomonoenol, tocodienol, or tocotrienol) bonded to (e.g., by an ester, amide, or thioester bond) to one or more polyethylene glycol (PEG) moieties via a linker (e.g., a dicarboxylic or tricarboxylic acid). The vitamin E moiety can be any naturally occurring or synthetic form of vitamin E, including α, β, γ, and δ isoforms, and all stereoisomers of tocopherol, tocomonoenol, tocodienol, and tocotrienol. Linkers include, for example, dicarboxylic acids (e.g., succinic acid, sebacic acid, dodecanedioic acid, suberic acid, or azelaic acid, citraconic acid, methylcitraconic acid, itaconic acid, maleic acid, glutaric acid, glutaconic acid, fumaric acids, and phthalic acids). Exemplary tocopherol polyethylene glycol diesters are D-alpha-tocopheryl PEG succinate, tocopherol sebacate polyethylene glycol, tocopherol dodecanodioate polyethylene glycol, tocopherol suberate polyethylene glycol, tocopherol azelaate polyethylene glycol, tocopherol citraconate polyethylene glycol, tocopherol methylcitraconate polyethylene glycol, tocopherol itaconate polyethylene glycol, tocopherol maleate polyethylene glycol, tocopherol glutarate polyethylene glycol, tocopherol glutaconate polyethylene glycol, and tocopherol phthalate polyethylene glycol. Each of the PEG moieties of the TPGS compound can be any polyethylene glycol or any PEG derivative, and can have a molecular weight of 200-6,000 kDa (e.g., 400-4,000 kDa, 500-2,000 kDa, 750-1,500 kDa, 800-1,200 kDa, 900-1,100 kDa, or 1,000 kDa). The PEG moieties can be polydisperse; that is, they can have a variety of molecular weights. PEG derivatives include, for example, methylated PEG, propylene glycol, PEG-NHS, PEG-aldehyde, PEG-SH, PEG-NH₂, PEG-CO₂H, PEG-OMe and other ethers, branched PEGs, and PEG copolymers (e.g., PEG-b-PPG-b-PEG-1100, PEG-PPG-PEG-1900, PPG-PEG-MBE-1700, and PPG-PEG-PPG-2000). Any known source of TPGS can be used in the present disclosure. An exemplary TPGS compound is tocopheryl PEG-1000 succinate (TPGS-1000), which has a PEG moiety having a molecular weight of 1,000 kDa. This TPGS is water-soluble form of natural-source vitamin E, which is prepared by esterification of crystalline D-α-tocopheryl acid succinate with polyethylene glycol 1000 (PEG 1000), and contains between 260 and 300 mg/g total tocopherol. Another exemplary TPGS compound is Water Soluble Natural Vitamin E (ZMC-USA, The Woodlands, Texas). Methods of preparing pegylated vitamin E are described in U.S. Pat. Nos. 2,680,749 and 3,102,078 and in U.S. Publication Nos. 2007/0184117 and 2007/0141203, which are herein incorporated by reference. TPGS compounds also include analogs that differ in chemical composition from tocopheryl PEG succinate (e.g., TPGS-1000) by the substitution, addition, or removal of one or more atoms, methylene (CH₂)ₙ units, or functional groups. TPGS compounds also include chromanol derivatives (e.g., 6-chromanol PEG-1000 succinate and 6-chromanol PEG-400 succinate), steroid derivatives (e.g., cholesteryl PEG-1000 succinate, cholic acid PEG-1000, dihydro cholic acid PEG-1000, litho-cholic acid PEG-1000, ursodeoxycholic acid PEG-1000, chenodeoxycholic acid PEG-1000), and others (e.g., indomethacin PEG-1000, chromone-2-carboxylic acid PEG-1000, chromone-2-carboxylic acid PEG-1100-OMe, chromone-2-carboxylic acid PEG-1500, chromone-2-carboxylic acid PEG-2000, naproxen PEG-1000, probenecid PEG-1000, 7-carboxymethoxy-4-methyl-coumarin PEG-1000, 5-(4-chlorophenyl)-2-furoic acid PEG-1000, probenecid tocopheryl PEG-1000 succinate, lithocholic acid PEG-1000, and chromone-3-carboxylic acid PEG-1000, 7-hydroxy-coumarinyl-4-acetic acid PEG-1000).

A “unit dosage form” means a unit of administration of an active agent, such as danicopan. Non-limiting examples of dosage forms include tablets, caplets, hard capsules, soft capsules, gelatin capsules, and liquid gelatin capsules.

As used herein, the term “administration” or “administering” refers to peroral (e.g., oral) administration of a drug to a subject or patient.

By “effective amount” is meant the amount of a drug sufficient to treat, prevent, or ameliorate a condition in a subject or patient. The effective amount of danicopan used to practice the methods disclosed herein for therapeutic management of a condition varies depending upon one or more of the age, body weight, sex, and/or general health or malady of the patient. The prescribers will primarily decide the appropriate amount and dosage regimen. Such amount is referred to as an “effective amount.”

As used herein, and as well understood in the art, “to treat” a condition or “treatment” of the condition (e.g., the conditions described herein) such as complement factor D mediated disorders (e.g., paroxysmal nocturnal hemoglobinuria (PNH), e.g., PNH with clinically evident extravascular hemolysis (EVH), and geographic atrophy (GA) secondary to age-related macular degeneration (AMD)) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in any suitable order unless otherwise indicated herein or otherwise clearly contradicted by context. The use of any and all examples, or exemplary language (e.g., “such as”), is intended merely for illustration and does not pose a limitation on the scope of the present disclosure unless otherwise claimed.

“Pharmaceutical compositions” are compositions including at least one active agent, such as danicopan, and at least one other substance, such as a carrier. Pharmaceutical compositions optionally contain more than one active agent.

The term “carrier” means a diluent, excipient, or vehicle with which a solid dispersion of danicopan is provided.

A “pharmaceutically acceptable excipient” means an excipient that is useful in preparing a pharmaceutical composition/combination that is generally safe, is sufficiently non-toxic, and neither biologically nor otherwise undesirable. A “pharmaceutically acceptable excipient” as used in the present application includes both one and more than one such excipient.

A “subject”, “patient”, or “host” is a human or non-human animal, including, but not limited to, simian, avian, feline, canine, bovine, equine or porcine in need of medical treatment. Medical treatment can include treatment of an existing condition, such as a disease or disorder, or a prophylactic or diagnostic treatment. In a particular embodiment, the patient or host is a human patient. In an alternative embodiment, the patient such as a host is treated to prevent a disorder or disease described herein.

The term “isolated” as used herein refers to the material in substantially pure form. An isolated compound does not have another component that materially affects the properties of the compound. In particular embodiments, an isolated form is at least 60, 70, 80, 90, 95, 98 or 99% pure.

Other features and advantages of the present disclosure will be apparent from the following detailed description, the drawings, and the claims.

### BRIEF DESCRIPTIONS OF THE DRAWINGS

FIG. 1 is a graph showing the XRPD diffractogram of the 80:20 danicopan/HPMCAS-H solid dispersion.

FIG. 2 is a graph of the dissolution kinetics assay of formulation 1 and formulation 2.

FIG. 3A is a graph of the pH shift dissolution kinetics of formulation 1.

FIG. 3B is a graph of the pH shift dissolution kinetics of formulation 2.

FIG. 4A. is a graph of the dissolution kinetics testing in 0.1N HCl at 37° C. and 50 rpm for Formulation 1A (F1A) at various tablet hardness levels (100 Newtons, 150 Newtons, and 200 Newtons).

FIG. 4B. is a graph of the dissolution kinetics testing in 0.1N HCl at 37° C. and 50 rpm for Formulation 1B (F2A) at various tablet hardness levels (100 Newtons, 150 Newtons, and 200 Newtons).

FIG. 4C. is a graph of the dissolution kinetics testing in 0.1N HCl at 37° C. and 50 rpm for Formulation 2A (F1B) at various tablet hardness levels (100 Newtons, 150 Newtons, and 200 Newtons).

FIG. 4D. is a graph of the dissolution kinetics testing in 0.1N HCl at 37° C. and 50 rpm for Formulation 2B (F2B) at various tablet hardness levels (100 Newtons, 150 Newtons, and 200 Newtons).

FIG. 5A is a graph of the supersaturation kinetics of the 70:30 danicopan/polymer solid dispersions and amorphous danicopan (labeled as “amorphous API”) in simulated intestinal fluid (SIF).

FIG. 5B is a graph of the supersaturation kinetics of the 80:20 danicopan/polymer solid dispersions and amorphous danicopan (labeled as “amorphous API”) in simulated intestinal fluid (SIF).

FIG. 5C is a graph of the supersaturation kinetics of the 85:15 danicopan/polymer solid dispersions and amorphous danicopan (labeled as “amorphous API”) in simulated intestinal fluid (SIF).

FIG. 5D is a graph of the supersaturation kinetics of the 90:10 danicopan/polymer solid dispersions and amorphous danicopan (labeled as “amorphous API”) in simulated intestinal fluid (SIF).

FIG. 6A is a graph of the supersaturation kinetics of the 70:30 danicopan/polymer solid dispersions and amorphous danicopan (labeled as “amorphous API”) in simulated gastric fluid (SGF).

FIG. 6B is a graph of the supersaturation kinetics of the 80:20 danicopan/polymer solid dispersions and amorphous danicopan (labeled as “amorphous API”) in simulated gastric fluid (SGF).

FIG. 6C is a graph of the supersaturation kinetics of the 85:15 danicopan/polymer solid dispersions and amorphous danicopan (labeled as “amorphous API”) in simulated gastric fluid (SGF).

FIG. 6D is a graph of the supersaturation kinetics of the 90:10 danicopan/polymer solid dispersions and amorphous danicopan (labeled as “amorphous API”) in simulated gastric fluid (SGF).

FIG. 7A is a graph of Cmax values of danicopan solid dispersion formulations in canines.

FIG. 7B is a graph of AUC₀₋₂₄ values of danicopan solid dispersion formulations in canines.

FIG. 8 is a graph of overlaid XRPD diffractograms of various danicopan solid dispersions in different polymer systems at 25% danicopan loading.

FIG. 9 is a graph of overlaid kinetic solubility profiles of various danicopan solid dispersions and crystalline danicopan in simulated gastric fluid (SGF) over 30 minutes, followed by conversion to fasted state simulated intestinal fluid (FaSSIF) over 20 min, at 37° C.

FIG. 10 is a graph of overlaid kinetic solubility profiles of various danicopan solid dispersions in simulated gastric fluid (SGF) over 30 minutes, followed by conversion to fasted state simulated intestinal fluid (FaSSIF) over 210 min, at 37° C.

FIG. 11A is a graph of overlaid XRPD diffractograms of the following spray dried solid dispersions: 40/60 danicopan/HPMCAS-M, 60/40 danicopan/HPMCAS-M, 80/20 danicopan/HPMCAS-M, 40/60 danicopan/HPMCAS-H, 60/40 danicopan/HPMCAS-H, and 80/20 danicopan/HPMCAS-H.

FIG. 11B is a graph of overlaid XRPD diffractograms of the following spray dried solid dispersions: 40/60 danicopan/PVP VA64, 60/40 danicopan/PVP VA64, 80/20 danicopan/PVP VA64, 40/60 danicopan/Eudragit, 60/40 danicopan/Eudragit, and 80/20 danicopan/Eudragit.

FIG. 12A is a graph of arithmetic mean plasma danicopan concentration-time profiles following administration of 200 mg danicopan as prototype PIC 1 fed and fasted and as tablet fed to healthy adult subjects (pharmacokinetic population) in a linear scale.

FIG. 12B is a graph of arithmetic mean plasma danicopan concentration-time profiles following administration of 200 mg danicopan as prototype PIC 1 fed and fasted and as tablet fed to healthy adult subjects (pharmacokinetic population) in a semi-log scale.

FIG. 13A is a graph of arithmetic mean plasma danicopan concentration-time profiles following administration of 200 mg danicopan as prototype PIC 2 fed and fasted and as tablet fed to healthy adult subjects (pharmacokinetic population) in a linear scale.

FIG. 13B is a graph of arithmetic mean plasma danicopan concentration-time profiles following administration of 200 mg danicopan as prototype PIC 2 fed and fasted and as tablet fed to healthy adult subjects (pharmacokinetic population) in a semi-log scale.

### DETAILED DESCRIPTION

The present disclosure provides formulations of danicopan and methods involving administration of said formulations to treat conditions related to complement Factor D mediated disorders. A non-limiting example of a condition treatable by this administration is paroxysmal nocturnal hemoglobinuria (PNH), e.g., PNH with clinically evident extravascular hemolysis (EVH), and geographic atrophy (GA) secondary to age-related macular degeneration (AMD).

Danicopan is a Factor D inhibitor that can treat various Factor D mediated disorders, and doses of up to 1,600 mg provided no adverse effects. It was previously discovered that danicopan can be prepared in a highly purified morphic forms that exhibit unexpectedly improved compound stability disclosed in PCT Application WO2020051538 assigned to Achillion Pharmaceuticals. The disclosed morphic forms of danicopan have improved stability that make them amenable to various pharmaceutical formulations and compositions. However, it was discovered that these morphic forms of danicopan exhibit poor oral bioavailability, making them unsuitable for therapeutic benefit (see, e.g., Table 9).

To overcome the poor bioavailability of the morphic forms, liquid soft gelatin capsule formulations of danicopan were developed. However, it was discovered that the liquid soft gelatin capsule formulation has limited shelf life, with large crystals of danicopan being detected in the soft gelatin capsule after one month of storage in a blister package (Example 2).

Accordingly, the present disclosure provides formulations to increase the oral bioavailability and/or to reduce patient-to-patient variability in pharmacokinetic behavior of danicopan. These formulations include use of a solid dispersion system with a polymer capable of maintaining danicopan in an amorphous form, thereby offering improved shelf stability over the liquid soft gelatin capsules.

### Solid Dispersion Pharmaceutical Compositions

### Pharmaceutically Acceptable Polymers for Making Solid Dispersions

Pharmaceutically acceptable polymers include one or more polymers capable of forming a solid dispersion with danicopan. Two or more polymers can be used to together and can optionally include one or more surfactants and/or plasticizers. Generally, optimal T_{g} values include from 50° C. to 180° C., which is higher than the melting temperature of danicopan but lower than the temperature at which danicopan decomposes. Exemplary T_{g} values for polymers are from 50° C. to 180° C. (e.g., from 50° C. to 85° C., from 50° C. to 80° C., from 50° C. to 75° C., from 50° C. to 70° C., from 50° C. to 65° C., from 50° C. to 60° C., from 75° C. to 100° C., from 75° C. to 95° C., from 75° C. to 90° C., from 75° C. to 85° C., from 75° C. to 80° C., from 80° C. to 110° C., from 80° C. to 105° C., from 80° C. to 100° C., from 80° C. to 95° C., from 80° C. to 90° C., from 80° C. to 85° C., from 85° C. to 125° C., from 85° C. to 120° C., from 85° C. to 115° C., from 85° C. to 110° C., from 85° C. to 105° C., from 85° C. to 100° C., from 85° C. to 95° C., from 85° C. to 90° C., from 90° C. to 125° C., from 90° C. to 120° C., from 90° C. to 115° C., from 90° C. to 110° C., from 90° C. to 105° C., from 90° C. to 100° C., from 90° C. to 95° C., from 95° C. to 125° C., from 95° C. to 120° C., from 95° C. to 115° C., from 95° C. to 110° C., from 95° C. to 105° C., from 95° C. to 100° C., from 100° C. to 135° C., from 100° C. to 130° C., from 100° C. to 125° C., from 100° C. to 120° C., from 100° C. to 115° C., from 100° C. to 110° C., from 100° C. to 105° C., from 110° C. to 150° C., from 110° C. to 145° C., from 110° C. to 140° C., from 110° C. to 135° C., from 110° C. to 130° C., from 110° C. to 125° C., from 110° C. to 120° C., from 110° C. to 115° C., from 120° C. to 145° C., from 120° C. to 140° C., from 120° C. to 135° C., from 120° C. to 130° C., from 120° C. to 125° C., from 125° C. to 150° C., from 125° C. to 145° C., from 125° C. to 140° C., from 125° C. to 135° C., from 125° C. to 130° C., from 130° C. to 160° C., from 130° C. to 150° C., from 130° C. to 145° C., from 130° C. to 140° C., from 130° C. to 135° C., from 135° C. to 150° C., from 135° C. to 145° C., from 135° C. to 140° C., from 150° C. to 180° C., from 150° C. to 170° C., from 150° C. to 160° C., or from 175° C. to 180° C.).

Exemplary T_{g} values for a solid dispersion including danicopan and one or more polymers are from 80° C. to 150° C. (e.g., from 80° C. to 145° C., from 80° C. to 140° C., from 80° C. to 135° C., from 80° C. to 130° C., from 80° C. to 125° C., from 80° C. to 120° C., from 80° C. to 115° C., from 80° C. to 110° C., from 80° C. to 105° C., from 80° C. to 100° C., from 80° C. to 95° C., from 80° C. to 90° C., from 80° C. to 85° C., from 85° C. to 150° C., from 85° C. to 145° C., from 85° C. to 140° C., from 85° C. to 135° C., from 85° C. to 130° C., from 85° C. to 125° C., from 85° C. to 120° C., from 85° C. to 115° C., from 85° C. to 110° C., from 85° C. to 105° C., from 85° C. to 100° C., from 85° C. to 95° C., from 85° C. to 90° C., from 90° C. to 150° C., from 90° C. to 145° C., from 90° C. to 140° C., from 90° C. to 135° C., from 90° C. to 130° C., from 90° C. to 125° C., from 90° C. to 120° C., from 90° C. to 115° C., from 90° C. to 110° C., from 90° C. to 105° C., from 90° C. to 100° C., from 90° C. to 95° C., from 95° C. to 150° C., from 95° C. to 145° C., from 95° C. to 140° C., from 95° C. to 135° C., from 95° C. to 130° C., from 95° C. to 125° C., from 95° C. to 120° C., from 95° C. to 115° C., from 95° C. to 110° C., from 95° C. to 105° C., from 95° C. to 100° C., from 120° C. to 150° C., from 120° C. to 145° C., from 120° C. to 140° C., from 120° C. to 135° C., from 120° C. to 130° C., from 120° C. to 125° C., from 125° C. to 150° C., from 125° C. to 145° C., from 125° C. to 140° C., from 125° C. to 135° C., from 125° C. to 130° C., from 130° C. to 150° C., from 130° C. to 145° C., from 130° C. to 140° C., from 130° C. to 135° C., from 135° C. to 150° C., from 135° C. to 145° C., and from 135° C. to 140° C.).

Exemplary polymers are one or more of ethyl cellulose (T_{g}=133° C.), cellulose acetate phthalate (CAP, T_{g}=171° C.), hydroxypropylmethyl cellulose acetate (T_{g}=177° C.), hydroxypropylmethyl cellulose acetate succinate (HPMCAS, T_{g}=115° C.), hydroxypropylmethyl cellulose phthalate (HPMCP, T_{g}=133° C.), polyvinyl pyrrolidone (T_{g}=174° C.), crospovidone (T_{g}=190° C. to 195° C.), polyvinyl alcohol (T_{g}=75° C.), and polyvinyl acetate phthalate (T_{g}=55° C.). Exemplary solid dispersions are those having 20% (w/w) of danicopan and CMEC (T_{g}=89.4° C.), HPMCAS-M (T_{g}=91.3° C.), CAP (T_{g}=129.9° C.), or PVAP (T_{g}=116.1° C.).

Examples of polymers which can be used in the formulations of the present disclosure are, without limitation, cellulose derivatives, polyacrylates, polyvinyl pyrrolidones, polyvinyl acetates, and copolymers thereof.

### Cellulose Derivatives

The formulations of the present disclosure can include one or more cellulose derivatives. Cellulose derivatives generally include those having any number of modifications to the free hydroxyl groups in cellulose.

In some examples, the cellulose derivative is a cellulose acetate having from 10% to 50% acetyl. Referring to cellulose derivatives, % refers to the proportion of the free hydroxyl groups esterified with a functional group. For example, “10% acetyl” refers to a derivative having 10% of the free hydroxyl groups in cellulose esterified with an acetyl group.

Particular examples of cellulose acetates are cellulose acetate phthalates (CAP), such as those having 35% phthalyl, 24% acetyl (available as Cellacefate); methylcellulose acetate phthalates; hydroxypropylmethyl cellulose acetates; and hydroxypropylmethyl cellulose acetate succinates (HPMCAS), such as M grade having 9% acetyl/11% succinoyl (e.g., HPMCAS having a mean particle size of 5 μm (i.e., HPMCAS-MF, fine powder grade) or having a mean particle size of 1 mm (i.e., HPMCAS-MG, granular grade)), H grade having 12% acetyl/6% succinoyl (e.g., HPMCAS having a mean particle size of 5 μm (i.e., HPMCAS-HF, fine powder grade) or having a mean particle size of 1 mm (i.e., HPMCAS-HG, granular grade)), and L grade having 8% acetyl/15% succinoyl (e.g., HPMCAS having a mean particle size of 5 μm (i.e., HPMCAS-LF, fine powder grade) or having a mean particle size of 1 mm (i.e., HPMCAS-LG, granular grade)).

Additional exemplary cellulose derivatives are alkyl celluloses, such as methyl cellulose (Methocel™ A) or ethylcellulose (Ethocel®); hydroxyalkyl celluloses, such as hydroxymethyl cellulose, hydroxyethyl cellulose, hydroxypropyl cellulose (HPC, e.g., low-substituted HPC having 11% hydroxypropyl or 8% hydroxypropyl), and hydroxybutyl cellulose; hydroxyalkylalkyl celluloses, such as hydroxyethylmethyl cellulose and hydroxypropylmethyl cellulose (hypromellose, HPMC, e.g., those having 19-24% methoxyl/7-12% hydroxypropyl (Methocel™ K, including those having apparent viscosity (2% in water at 20° C.) of 80-120 cP (Methocel™ K100), 3,000-5,600 cP (Methocel™ K4M), 11,250-21,000 cP (Methocel™ K15M), 80,000-120,000 cP (Methocel™ K100M)), 28-30% methoxyl/7-12% hydroxypropoxyl (Methocel™ E, including those having apparent viscosity (2% in water at 20° C.) of 3,000-5,600 cP (Methocel™ E4M) and 7,500-14,000 cP (Methocel™ E10M), also available from Dow Chemical Co.), 23% methoxyl/10% hydroxypropyl (Metolose® SR, available from Shin-Etsu Chemical Co., Ltd., Tokyo, Japan), 23%-29% methoxyl/8%-9% hydroxypropyl (Metolose®, also available from Shin-Etsu Chemical Co., Ltd.), 29% methoxyl/9% hydroxypropyl (Hypromellose USP, substitution 2910), and 23% methoxyl/6% hydroxypropyl (Hypromellose USP, substitution 2208)); hydroxyalkylalkyl cellulose esters, such as hydroxypropylmethyl cellulose phthalate (HPMCP) (e.g., HP 55 grade having 31% nominal phthalyl content and HP-55S or HP-50 grades having 24% nominal phthalyl content); carboxyalkyl celluloses, such as carboxymethyl cellulose and alkali metal salts thereof, such as sodium salts; carboxyalkylalkyl celluloses, such as carboxymethylethyl cellulose; and carboxyalkyl cellulose esters, such as carboxymethyl cellulose butyrate, carboxymethyl cellulose propionate, carboxymethyl cellulose acetate butyrate, and carboxymethyl cellulose acetate propionate. Any of the cellulose derivatives herein can be further cross-linked or copolymerized (e.g., with any polymer described herein).

### Polyacrylates

The formulations of the present disclosure can include one or more polyacrylates or copolymers thereof. Exemplary polyacrylates are polymethacrylates; methacrylate copolymers, such as methacrylic acid-methyl methacrylate copolymers having a 1:1 ratio of free carboxyl groups to ester groups (e.g., Eudragit® L 100, MW ~125,000 Da) and a 1:2 ratio of free carboxyl groups to ester groups (Eudragit® S 100, MW ~125,000 Da), dimethylaminoethyl methacrylate-butyl methacrylate-methyl methacrylate copolymers (e.g., having a ratio of 2:1:1 of dimethylaminoethyl methacrylate-butyl methacrylate-methyl methacrylate and in powder, granule, or solution forms, i.e., Eudragit® E PO, Eudragit® E 100, or Eudragit® E 12.5, respectively), and diethylaminoethyl methacrylic acid-methyl methacrylate copolymers (e.g., Eudragit® E); and ethacrylate copolymers, such as methacrylic acid ethacrylate copolymers having a 50:50 ratio of methacrylic acid to ethacrylate (e.g., Kollicoat® MAE 100P or Eudragit®L 100-55, MW ~320,000 Da).

### Polyvinyl Pyrrolidones and Polyvinyl Acetates

The formulations of the present disclosure can include one or more polyvinyl pyrrolidones, polyvinyl acetates, or copolymers thereof.

Exemplary polyvinyl pyrrolidones and polyvinyl acetates are polyvinyl pyrrolidones (e.g., povidone, PVP, PVP K30, or soluble povidone) having molecular weights of 2,500 Da (Kollidon®12 PF, weight-average molecular weight between 2,000 to 3,000 Da), 9,000 Da (Kollidon®17 PF, weight-average molecular weight between 7,000 to 11,000 Da), 25,000 Da (Kollidon®25, weight-average molecular weight between 28,000 to 34,000 Da), 50,000 Da (Kollidon®30, weight-average molecular weight between 44,000 to 54,000 Da), and 1,250,000 Da (Kollidon®90 or Kollidon®90F, weight-average molecular weight between 1,000,000 to 1,500,000 Da); polyvinyl acetate esters, such as polyvinyl acetate phthalate (PVAP); polyethylene glycol-polyvinyl acetate copolymers, such as polyethylene glycol-polyvinylcaprolactam-polyvinylacetate copolymer (Soluplus®); and polyvinyl pyrrolidone-polyvinyl acetate copolymers (PVP VA), such as those having a 60:40 ratio of N-vinyl-2-pyrrolidone to vinyl acetate (copovidone, also available as Kollidon® VA 64 or PVP VA64) and a 20:80 ratio of N-vinyl-2-pyrrolidone to vinyl acetate (Kollidon® SR).

### Methods of Making Solid Dispersion Systems

The pharmaceutical composition including the solid dispersion (e.g., a spray dried dispersion (SDD) or a hot melt extrusion (HME)) can be made using any useful method. Generally, one or more polymers and danicopan, are combined either with or without a solvent (e.g., one or more of dimethyl acetamide, dimethyl formamide, pyrrolidone, methyl pyrrolidone, methanol, ethanol, dichloromethane, and acetone) to form a mixture (e.g., a liquid mixture) or a solution. Optionally, the polymer and danicopan, either with or without additional excipients, can be heated near or past the glass transition temperature T_{g} or melting temperature Tₘ to form a liquid mixture. Then, the resultant solution can be spray dried to form a solid dispersion. Alternatively, the method includes a hot-melt extrusion process, where the mixture is heated to form a homogenous molten mass, extruded, and cooled to form a solid dispersion. The extrudates can optionally be pelletized or milled to form a solid dispersion amenable for further processing in a suitable unit dosage form.

### Spray Dried Processes

The compositions of the present disclosure can be prepared by any useful process, such as spray drying to form a spray dried dispersion (SDD). In one example, one or more polymers and danicopan are combined with one or more solvents (e.g., acetone) to form a solution having 4% (w/w) to 80% (w/w) of total solids. Percentage (w/w) total solids is determined by dividing the total mass of the compound and one or more polymers by the total mass of the compound, one or more polymers, and one or more solvents. The solution can then be spray dried to form a SDD, which can optionally undergo further drying steps. In particular embodiments, the SDD includes 80% (w/w) of danicopan with the one or more polymers (i.e., the weight ratio of danicopan to the polymer is 4:1).

For example, to produce an 80% (w/w) danicopan in a SDD, a solution was prepared having 8% (w/w) danicopan and 2% (w/w) of a pharmaceutically acceptable polymer or a combination of a pharmaceutically acceptable polymer in acetone. The solution was then spray dried at the appropriate temperature (e.g., between 40° C. to 62° C. for HPMCAS at the appropriate solution flow rate).

The resultant SDD can be blended with one or more excipients, as described herein, and then granulated and/or compacted to produce a final blend for encapsulating or tableting. In particular embodiments, the one or more excipients include a binding agent, a filler, a disintegrating agent, a wetting agent, a glidant, and a lubricant.

### Other Pharmaceutically Acceptable Excipients

Pharmaceutically acceptable excipients useful in the formulations of the present disclosure can further include one or more other ingredient capable of maintaining danicopan in a substantially amorphous form. In particular embodiments, the excipient is a pharmaceutically acceptable polymer, as described herein.

Exemplary excipients include antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Additional non-limiting exemplary excipients include colorants, flavoring agents, plasticizers, humectants, and buffering agents. In some embodiments, the excipients include one or more of lactose monohydrate (e.g., Tablettose 80), fumed silica (e.g., Aerosil), magnesium stearate, croscarmellose sodium, and microcrystalline cellulose (e.g., Avicel PH101 and/or Avicel PH102).

The pharmaceutical formulations can include the solid dispersion of danicopan described herein, in any pharmaceutically acceptable carrier. The solid dispersion is used for filling any one of the unit dosage forms described herein (e.g., a capsule) or for tableting. The solid dispersion can optionally be further processed before filling or tableting. Exemplary further processing includes spheronizing, pelletizing, milling, injection molding, sieving, and/or calendaring the solid dispersion. In one embodiment a solid dispersion as described herein is administered to a patient in need thereof as a spray dried dispersion (SDD). In another embodiment the present disclosure provides a spray dried dispersion (SDD) including a danicopan and one or more pharmaceutically acceptable excipients as defined herein. In a further embodiment the SDD includes a danicopan and one or more pharmaceutically acceptable excipients. In another embodiment any of the described spray dried dispersions can be coated to form a coated tablet. In an alternative embodiment the spray dried dispersion is formulated into a tablet but is uncoated.

### Surfactants

The formulations of the present disclosure optionally include one or more surfactants. Exemplary surfactants are liquid and solid polyethoxylated esters of fatty acids, such as polyoxyl 40 stearate (Myrj® 52, hydrophobic-lipophilic balance (“HLB”)=17), PEG 400 monostearate, also known as polyoxyl 8 stearate (Myrj® 45, HLB=11), and PEG 660 hydroxystearate, also known as PEG 15 hydroxystearate (Solutol® HS15, HLB=14 to 16); polyethoxylated alkyl ethers, such as polyoxyl 10 oleyl ether (Brij® 97, HLB=12.4) and PEG 25 cetostearyl ether (Cremophor® A 25, HLB=15 to 17); polyethoxylated sorbitan esters, such as polysorbate 20 (Tween® 20, HLB=15) and polysorbate 80 (Tween® 80, HLB=11.5); polyethoxylated glyceryl esters having high HLB values (e.g., from 10 to 20), such as polyoxyl 35 castor oil (Cremophor® EL, HLB=12 to 14) and polyoxyl 40 castor oil having 40-45 moles of ethylene oxide (Cremophor® RH-40, HLB=14 to 16); polyethoxylated glyceryl esters of fatty acids having high HLB values (e.g., from 10 to 20), such as a mixture of PEG 6 caprylic/capric glyceryl esters having <2% C₆/50%-80% C₈/20%-50% C₁₀/<3% C₁₂/<1% C₁₄ (Softigen® 767, HLB=19), a mixture of PEG 8 caprylic/capric glyceryl esters having 50%-80% C₈/20%-50% C₁₀/<3% C₁₂/<1% C₁₈ (Labrasol®, HLB=14), a mixture of PEG 32 lauryl glyceryl esters having 40%-50% C₁₂/14%-24% C₁₄/4%-10% C₈/3-9% C₁₀/4%-14% C₁₆/5%-15% C₁₈ (Gelucire® 44/14, HLB=14), and a mixture of PEG 32 stearyl glyceryl esters having 40%-50% C₁₆/48%-58% C₁₈ (Gelucire® 50/13, HLB=13); polyethoxylated vitamin analogs, such as D-alpha-tocopheryl PEG 1000 succinate (HLB=13); and ethoxylated propoxylated block copolymers having formula H(OCH₂CH₂)ₐ(OCHCH₃CH₂)_{b}(OCH₂CH₂)ₐOH, where a is 12 and b is 20 (Poloxamer® 124), where a is 38 and b is 29, where a is 80 and b is 27 (Poloxamer® 188), where a is 64 and b is 37 (Poloxamer® 237), where a is 141 and b is 44 (Poloxamer® 338), where a is 49 and b is 57, and where a is 101 and b is 56 (Poloxamer® 407).

### Plasticizers

The formulations of the present disclosure optionally include one or more plasticizers. Generally, plasticizers can be used to reduce the glass transition temperature T_{g} or to decrease viscosity of the mixture of danicopan with the polymer. Exemplary plasticizers are polyalkylene oxides, such as polyethylene glycols (e.g., PEG 300, PEG 400, PEG 4000, or PEG 8000) and polypropylene glycols; copolymers of ethylene oxide and propylene oxide, such as ethoxylated propoxylated block copolymers having the formula H(OCH₂CH₂)ₐ(OCHCH₃CH₂)_{b}(OCH₂CH₂)ₐOH, where a is 12 and b is 20 (Poloxamer® 124), where a is 38 and b is 29, where a is 80 and b is 27 (Poloxamer® 188), where a is 64 and b is 37 (Poloxamer® 237), where a is 141 and b is 44 (Poloxamer® 338), where a is 49 and b is 57, and where a is 101 and b is 56 (Poloxamer® 407); and polyethoxylated glyceryl esters, such as polyoxyl 35 castor oil (Cremophor® EL, HLB=12 to 14) and polyoxyl 40 castor oil having 40-45 moles of ethylene oxide (Cremophor® RH-40, HLB=14 to 16).

### Hot Melt Extrusion Processes

In some embodiments, the compositions of the present disclosure are prepared by hot melt extrusion (HME). In one example, one or more polymers and danicopan are combined to form a mixture, where this mixture can optionally include a surfactant. The mixture can then be fed into a pre-heated extruder (e.g., an extruder having temperature zones between 75° C. to 145° C.) to produce an initial extrudate. The extrudate is then pelletized and milled (e.g., to a size less than 500 μm) to produce a fine milled extrudate.

For example, to produce a 20% (w/w) danicopan extrudate, a pre-blend was prepared having 20% (w/w) danicopan and 80% (w/w) of a pharmaceutically acceptable polymer or a combination of a pharmaceutically acceptable polymer with a surfactant (e.g., any pharmaceutically acceptable polymer and/or surfactant described herein). Any component of the pre-blend can be pre-milled or pre-sieved. For example, the pharmaceutically acceptable polymer and/or surfactant can be milled through a bar rotor and rasping screen to reduce particle size (e.g., reduce down to ≤600 microns); and/or danicopan can be pre-sieved. Then, the pre-blend was processed using a co-rotating twin screw extruder, and the resultant extrudate was processed further by milling (pelletizing) to reduce its particle size (e.g., ≤500 microns). The milled/pelletized extrudate was sieved and blended with various pharmaceutically acceptable excipients (e.g., any described herein), where the resultant blend was then co-milled. The co-milled blend can be further processed by adding a lubricant (e.g., magnesium stearate), and the resultant, processed blend can be used to fill a unit dosage form (e.g., a capsule).

Polymers for hot melt extrusion include a cellulose derivative, such as HPMCAS, e.g., grades L, H, and M; a polyvinyl pyrrolidone (PVP), such as povidone having a molecular weight of 50,000 Da (Kollidon®30, weight-average molecular weight between 44,000 to 54,000 Da); a polyvinyl acetate; or a copolymer of a polyvinyl pyrrolidone and a polyvinyl acetate (PVP-VA), such as those having a 60:40 ratio of N-vinyl-2-pyrrolidone to vinyl acetate (copovidone, also available as Kollidon® VA 64) and a 20:80 ratio of N-vinyl-2-pyrrolidone to vinyl acetate (Kollidon® SR).

In particular embodiments, any surfactant or wetting agent described herein can be included in the mixture to enhance dissolution and/or enhance stability. An exemplary surfactant includes a TPGS compound, such as any described herein (e.g., D-alpha-tocopheryl PEG 1000 succinate), in a useful amount (e.g., from 3% to 10% (w/w), e.g., 5% (w/w)).

The resultant extrudate can be blended with one or more excipients, as described herein, and then milled, blended, granulated and/or compacted to produce a final blend for encapsulating or tableting. In particular embodiments, the one or more excipients include a binding agent, a filler, a surfactant (e.g., a TPGS compound), a disintegrating agent, a wetting agent, a glidant, and a lubricant.

### Liquid Gelatin Capsule Pharmaceutical Compositions

### Glycerides for Formulations

Glycerides can be used in the pharmaceutical compositions of the liquid gelatin capsule formulation of danicopan. Glycerides are fatty acid mono-, di-, and tri-esters of glycerol. Glycerides include saturated and unsaturated monoglycerides, diglycerides (1,2- and 1,3-diglycerides), and triglycerides, with mixed and unmixed fatty acid composition. Each glyceride is herein designated as (Cn:m), where n is the length of the fatty acid side chain and m is the number of double bonds (cis- or trans-) in the fatty acid side chain. Examples of commercially available monoglycerides include: monocaprylin (C8; i.e., glyceryl monocaprylate) (Larodan), monocaprin (C10; i.e., glyceryl monocaprate) (Larodan), monolaurin (C12; i.e., glyceryl monolaurate) (Larodan), monopalmitolein (C16:1) (Larodan), glyceryl monomyristate (C14) (Nikkol® MGM, Nikko), glyceryl monooleate (C18:1) (PECEOL®, Gattefosse), glyceryl monooleate (Myverol®, Eastman), glycerol monooleate/linoleate (OLICINE®, Gattefosse), glycerol monolinoleate (Maisine, Gattefosse), and monoelaidin (C18:1) (Larodan). Examples of commercially available mono/di and tri glycerides include Capmul MCM C8EP, (C8:C10 mono/di glycerides) and Capmul MCM (mono/di glycerides). Examples of commercially available diglycerides include glyceryl laurate (Imwitor® 312, Huls), glyceryl caprylate/caprate (Capmul® MCM, ABITEC), caprylic acid diglycerides (Imwitor® 988, Huls), caprylic/capric glycerides (Imwitor® 742, Huls), dicaprylin (C8) (Larodan), dicaprin (C10) (Larodan), dilaurin (C12) (Larodan), glyceryl dilaurate (C12) (Capmul® GDL, ABITEC®). Examples of commercially available triglycerides include: tricaprylin (C8; i.e., glyceryl tricaprylate) (Larodan), capatex 100 (C10), tricaprin (C10; i.e., glyceryl tricaprate) (Larodan), trilaurin (C12; i.e., glyceryl trilaurate) (Larodan), dimyristin (C14) (Larodan), dipalmitin (C16) (Larodan), distearin (Larodan), glyceryl dilaurate (C12) (Capmul® GDL, ABITEC), glyceryl dioleate (Capmul® GDO, ABITEC®), glycerol esters of fatty acids (GELUCIRE® 39/01, Gattefosse), dipalmitolein (C16:1) (Larodan), 1,2 and 1,3-diolein (C18:1) (Larodan), dielaidin (C18:1) (Larodan), and dilinolein (C18:2) (Larodan).

### TPGS Compounds

D-α-Tocopheryl polyethylene glycol succinate (tocopheryl PEG-1000 succinate) and related TPGS compounds can be used in the liquid gelatin capsule formulations of the present disclosure. Tocopheryl PEG-1000 succinate has the following structure:

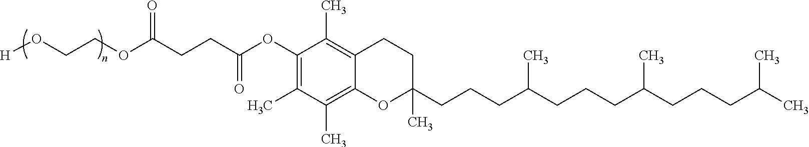

where n is an integer.

Related TPGS compounds include additives formed using different diacid linkers, different length polyethylene glycol tails, and different isoforms (e.g., α-, β-, γ-, or δ-) of tocopherol, tocomonoenol, tocodienol, and tocotrienol. These include α-tocopherol, α-tocomonoenol, α -tocodienol, α-tocotrienol, β-tocopherol, β-tocomonoenol, β-tocodienol, β-tocotrienol, γ-tocopherol, γ-tocomonoenol, γ-tocodienol, γ-tocotrienol, δ-tocopherol, δ-tocomonoenol, δ-tocodienol, δ-tocotrienol, and any stereoisomer thereof. Suitable vitamin E compounds of the present disclosure also include desmethyl-tocopherol, desmethyl-tocomonoenol, desmethyl-tocodienol, desmethyl-tocotrienol, and any stereoisomer thereof. Furthermore, when a compound disclosed herein contains one or more chiral atoms where stereochemistry is unspecified, it will be understood that each stereoisomer of the compound is individually disclosed as if the structure of each stereoisomer were explicitly drawn. In certain embodiments of the present disclosure, the vitamin E compound may be a naturally-occurring δ-stereoisomer of vitamin E.

The vitamin E moieties of the present disclosure may be naturally occurring or synthetic. Certain embodiments of the present disclosure include a naturally occurring vitamin E compound such as an extract from a food source. For example, α-tocopherol, α-tocotrienol, β-tocopherol, β-tocotrienol, γ-tocopherol, γ-tocotrienol, δ-tocopherol, and δ-tocotrienol are available naturally from fortified cereals, green vegetables, nuts, seeds, and vegetable oils. Methods of extracting vitamin E from natural sources have been described, for example, in U.S. Pat. Nos. 6,743,450; 6,838,104; 7,161,055; and 7,544,822, which are hereby incorporated by reference.

The TGPS compound can include synthetic vitamin E moieties. An exemplary method for making α-tocopherol is the reaction of trimethylhydroquinone (TMHQ) with iso-phytol (3,7,11,15-tetramethylhexadec-1-en-3-ol) in a condensation reaction with a catalyst. It will be apparent to one skilled in the art that other tocopherol, tocomonoenol, tocodienol, and tocotrienol isoforms and their derivates can also be prepared using a similar strategy starting from appropriate precursors. For example, the starting compounds may be TMHQ and 3,7,11,15-tetramethylhexadec-2-en-1-ol. An additional method of making vitamin E with isophytol under relatively mild conditions has been described by Wehrli et al., J. Org. Chem. 36:2910 (1971). Methods for synthesizing unsaturated side chains of vitamin E are described in U.S. Pat. No. 4,168,271, which is hereby incorporated by reference. Additional methods of synthesizing vitamin E side chains have been reviewed by Stalla-Bourdillon, Ind. Chim. Belg. 35, 13 (1970). Additional methods of synthesizing tocopherols are described in U.S. Pat. Nos. 5,523,420, and 6,005,122, each of which is incorporated herein by reference. Additional methods of synthesizing tocotrienols are described in U.S. Pat. No. 7,038,067, which is hereby incorporated by reference.

The TGPS compounds can include different linkers, for example, dicarboxylic acids (e.g., succinic acid, sebacic acid, dodecanedioic acid, suberic acid, or azelaic acid, citraconic acid, methylcitraconic acid, itaconic acid, maleic acid, glutaric acid, glutaconic acid, fumaric acids and phthalic acids). Exemplary tocopherol polyethylene glycol diesters are TPGS, tocopherol sebacate polyethylene glycol, tocopherol dodecanodioate polyethylene glycol, tocopherol suberate polyethylene glycol, tocopherol azelaate polyethylene glycol, tocopherol citraconate polyethylene glycol, tocopherol methylcitraconate polyethylene glycol, tocopherol itaconate polyethylene glycol, tocopherol maleate polyethylene glycol, tocopherol glutarate polyethylene glycol, tocopherol glutaconate polyethylene glycol, and tocopherol phthalate polyethylene glycol.

The PEG moiety of the TPGS compound can be any polyethylene glycol or derivative thereof, and can have a molecular weight of 200-6000 kDa (e.g., 400-4000 kDa, 500-2000 kDa, 750-1500 kDa, 800-1200 kDa, 900-1100 kDa, or 1000 kDa). PEG derivatives include, for example, methylated PEG, polypropylene glycol (PPG), PEG-NHS, PEG-aldehyde, PEG-SH, PEG-NH₂, PEG-CO₂H, PEG-OMe and other ethers, branched PEGs, and PEG copolymers (e.g., PEG-b-PPG-b-PEG-1100, PEG-PPG-PEG-1900, PPG-PEG-MBE-1700, and PPG-PEG-PPG-2000).

Any known source of TPGS compound can be used in the present disclosure. TPGS typically has an HLB value between 13 and 18. An exemplary TPGS compound is tocopheryl PEG-1000 succinate (also referred to herein as “TPGS 1000”), which has a PEG moiety having a molecular weight of 1000 kDa. A food grade TPGS 1000 is available, for example, under the trade name Eastman Vitamin E TPGS® (Eastman Chemical Company, Kingsport, Tenn.). This TPGS is water-soluble form of natural-source vitamin E, which is prepared by esterification of crystalline D-α-tocopheryl acid succinate with polyethylene glycol 1000 (PEG 1000), and contains between 260 and 300 mg/g total tocopherol. Another exemplary TPGS compound is Water Soluble Natural Vitamin E (ZMC-USA, The Woodlands, Tex.). Methods of preparing TPGS are described in U.S. Pat. Nos. 2,680,749 and 3,102,078 and in U.S. Publication Nos. 2007/0184117 and 2007/0141203, which are herein incorporated by reference.

TPGS analogs also include chromanol derivatives (e.g., 6-chromanol PEG-1000 succinate and 6-chromanol PEG-400 succinate), steroid derivatives (e.g., cholesteryl PEG-1000 succinate, cholic acid PEG-1000, dihydro cholic acid PEG-1000, litho-cholic acid PEG-1000, ursodeoxycholic acid PEG-1000, chenodeoxycholic acid PEG-1000), and others (e.g., indomethacin PEG-1000, chromone-2-carboxylic acid PEG-1000, chromone-2-carboxylic acid PEG-1100-OMe, chromone-2-carboxylic acid PEG-1500, chromone-2-carboxylic acid PEG-2000, naproxen PEG-1000, probenecid PEG-1000, 7-carboxymethoxy-4-methyl-coumarin PEG-1000, 5-(4-chlorophenyl)-2-furoic acid PEG-1000, probenecid tocopheryl PEG-1000 succinate, lithocholic acid PEG-1000, and chromone-3-carboxylic acid PEG-1000, 7-hydroxy-coumarinyl-4-acetic acid PEG-1000)

Pharmaceutically acceptable organic solvents can be used in the liquid gelatin capsule formulations of the present disclosure. Pharmaceutically acceptable organic solvents include propylene glycol, ethanol, N-methyl pyrrolidone, or glycerol.

### Unit Dosage Forms

For use as treatment of human and animal subjects, danicopan can be formulated as pharmaceutical or veterinary compositions. Depending on the subject to be treated, the mode of administration, and the type of treatment desired (e.g., prevention, prophylaxis, or therapy) the compounds are formulated in ways consistent with these parameters. Exemplary techniques for preparing pharmaceutical compositions are found in Remington: The Science and Practice of Pharmacy, 21st Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Danicopan may be present in amounts totaling 10-95% by weight of the total weight of the composition. The composition including danicopan and a pharmaceutically acceptable polymer may be provided in a dosage form that is suitable for oral administration. Alternatively, the unit dosage form of the present disclosure includes substantially amorphous danicopan and a pharmaceutically acceptable excipient (e.g., fillers, diluents, lubricants, and/or glidants). Thus, the pharmaceutical composition may be in the form of, e.g., hard capsules (e.g., hard gelatin capsules or hard hydroxypropyl methylcellulose capsules), soft gelatin capsules, tablets, caplets, enteric coated tablets, chewable tablets, enteric coated hard gelatin capsules, enteric coated soft gelatin capsules, minicapsules, lozenges, films, strips, gelcaps, dragees, suspensions, syrups, or sprinkles. The compositions may be formulated according to conventional pharmaceutical practice.

In particular embodiments, danicopan and a pharmaceutically acceptable polymer are included in a capsule or compressed into a tablet. Danicopan in combination with a pharmaceutically acceptable polymer can be in any form, such as a semi-solid suspension, a solid suspension, a homogenous melt, solid particles, or semi-solid particles. The form of the danicopan can be determined based on dose. For example, a capsule filled with a solid dispersion can be used for approximately 10-95% drug loading.

Exemplary unit dosage forms are encapsulated powders, hard capsules (e.g., hard gelatin capsules or hard hydroxypropyl methylcellulose capsules), tablets, and soft gelatin capsules (e.g., liquid soft gelatin capsules). When soft gelatin capsules are used, it is preferred that when a composition contains propylene glycol or a polyethylene glycol, and the composition of the soft gelatin capsule shell contains a humectant, for example, sorbitol, to prevent brittleness of the soft gelatin capsule.

### Uses of Danicopan for Treatment of Selected Disorders

In one aspect, an effective amount of danicopan formulated as a solid dispersion as described herein is used to treat a medical disorder which is an inflammatory or immune condition, a disorder mediated by the complement cascade (including a dysfunctional cascade) including a complement factor D-related disorder or alternative complement pathway-related disorder, a disorder or abnormality of a cell that adversely affects the ability of the cell to engage in or respond to normal complement activity, or an undesired complement-mediated response to a medical treatment, such as surgery or other medical procedure or a pharmaceutical or biopharmaceutical drug administration, a blood transfusion, or other allogenic tissue or fluid administration.

In one embodiment, a method for the treatment of paroxysmal nocturnal hemoglobinuria (PNH) is provided that includes the administration of an effective amount of a solid dispersion formulation described herein, optionally in a pharmaceutically acceptable composition. PNH is a non-malignant, hematological disorder characterized by the expansion of hematopoietic stem cells and progeny mature blood cells that are deficient in some surface proteins. PNH erythrocytes are not capable of modulating their surface complement activation, which leads to the typical hallmark of PNH—the chronic activation of complement mediated intravascular anemia.

In a further embodiment, a method for the treatment of PNH with clinically evident extravascular hemolysis (EVH) that includes the administration of an effective amount of a solid dispersion formulation described herein, optionally in a pharmaceutically acceptable composition. In a further embodiment, a method for the treatment of PNH with EVH is provided that includes the administration of an effective amount of a solid dispersion formulation described herein, optionally in a pharmaceutically acceptable composition, in combination with a complement component 5 (C5) inhibitor (e.g., an anti-C5 antibody such as eculizumab or ravulizumab).

In another embodiment, a method for the treatment of age-related macular degeneration (AMD) (e.g., wet AMD, dry AMD, or intermediate AMD) in a host is provided that includes the administration of an effective amount of a solid dispersion formulation described herein, optionally in a pharmaceutically acceptable composition. AMD is a leading cause of vision loss in industrialized countries. Based on a number of genetic studies, there is evidence of the link between the complement cascade and macular degeneration. Individuals with mutations in the gene encoding complement Factor H have a fivefold increased risk of macular degeneration and individuals with mutations in other complement factor genes also have an increased risk of AMD. Individuals with mutant Factor H also have increased levels of C-reactive protein, a marker of inflammation. Without adequate functioning of Factor H, the alternative pathway of the complement cascade is overly activated leading to cellular damage. In a further embodiment, a method for the treatment of geographic atrophy (GA) secondary to AMD in a host is provided that includes the administration of an effective amount of a solid dispersion formulation described herein, optionally in a pharmaceutically acceptable composition.

Other disorders that have been linked to the complement cascade include acute respiratory distress syndrome (ARDS), arthritis, asthma, Alzheimer's dementia, amyotrophic lateral sclerosis (ALS), antibody-mediated transplant rejection, antineutrophil cytoplasm antibody-associated vasculitis, antiphospholipid syndrome, atypical hemolytic uremic syndrome (aHUS) or hemolytic uremic syndrome (HUS), chronic obstructive pulmonary disease (COPD), cirrhosis, cold agglutinin disease, complement 3 (C3) glomerulopathy (e.g., C3 glomerulonephritis or dense deposit disease), diabetic macular edema, diabetic retinopathy, dermatomyositis, dermatitis, epidermolysis bullosa acquisita, fatty liver, focal segmental glomerulosclerosis, glomerulonephritis, graft versus host disease, Guillain Barre syndrome, hemolytic anemia, hidradenitis suppurativa, IgA nephropathy, ischemia/reperfusion injury, liver failure, liver inflammation, lupus nephritis, membrane proliferative glomerulonephritis, multifocal motor neuropathy, multiple sclerosis, myasthenia gravis (MG), neuromyelitis optica (NMO), nonalcoholic steatohepatitis (NASH), pemphigoid, pemphigus vulgaris, pre-eclampsia, reduced glomerular filtration rate, respiratory disease, retinal detachment, retinopathy of prematurity, rheumatoid arthritis, scleroderma, sepsis, Shiga toxin E. coli-related hemolytic uremic syndrome, spinal cord injury, sickle cell disease, systemic lupus erythematosus (SLE), traumatic brain injury, ulcerative colitis, and uveitis. Accordingly, the present disclosure provides a method of treating any one of such disorders by administering to a subject in need thereof an effective amount of a solid dispersion formulation described herein, optionally in a pharmaceutically acceptable composition.

### Dosage

In some embodiments, the pharmaceutical composition disclosed herein contains 20 mg to 900 mg of danicopan. In some embodiments, the pharmaceutical composition disclosed herein contains 100 mg to 400 mg of danicopan. In some embodiments, the pharmaceutical composition disclosed therein contains 100 mg to 200 mg of danicopan. In some embodiment, the pharmaceutical composition disclosed herein contains 100 mg of danicopan. In some embodiments, the pharmaceutical composition disclosed herein contains 50 mg of danicopan. In one embodiment, the pharmaceutical composition is formulated as a capsule and contains 50 mg to 900 mg (e.g., 50 mg to 75 mg, 50 mg to 100 mg, 50 mg to 125 mg, 50 mg to 150 mg, 50 mg to 175 mg, 50 mg to 200 mg, 50 mg to 225 mg, 50 mg to 250 mg, 60 mg to 75 mg, 60 mg to 100 mg, from 60 mg to 125 mg, from 60 mg to 150 mg, from 60 mg to 175 mg, from 60 mg to 200 mg, 60 mg to 225 mg, from 60 mg to 250 mg, 70 mg to 75 mg, 70 mg to 100 mg, 70 mg to 125 mg, 70 mg to 150 mg, 70 mg to 175 mg, 70 mg to 200 mg, 70 mg to 225 mg, 70 mg to 250 mg, 80 mg to 100 mg, 80 mg to 125 mg, 80 mg to 150 mg, 80 mg to 175 mg, 80 mg to 200 mg, 80 mg to 225 mg, 80 mg to 250 mg, 90 mg to 100 mg, 90 mg to 125 mg, 90 mg to 150 mg, 90 mg to 175 mg, 90 mg to 200 mg, from 90 mg to 225 mg, from 90 mg to 250 mg, from 100 mg to 125 mg, from 100 mg to 150 mg, from 100 mg to 175 mg, from 100 mg to 200 mg, from 100 mg to 225 mg, from 100 mg to 250 mg, 200 mg to 225 mg, from 200 mg to 250 mg, from 200 mg to 275 mg, from 200 mg to 300 mg, from 200 mg to 325 mg, and from 200 mg to 350 mg, 300 mg to 325 mg, from 300 mg to 350 mg, from 300 mg to 375 mg, from 300 mg to 400 mg, from 300 mg to 425 mg, from 300 mg to 450 mg, from 400 mg to 425 mg, from 400 mg to 450 mg, from 400 mg to 475 mg, from 400 mg to 500 mg, from 400 mg to 525 mg, from 400 mg to 550 mg, from 500 mg to 525 mg, from 500 mg to 550 mg, from 500 mg to 575 mg, from 500 mg to 600 mg, from 500 mg to 625 mg, from 500 mg to 650 mg, from 600 mg to 625 mg, from 600 mg to 650 mg, from 600 mg to 675 mg, from 600 mg to 700 mg, from 600 mg to 725 mg, from 600 mg to 750 mg, from 700 mg to 725 mg, from 700 mg to 750 mg, from 700 mg to 775 mg, from 700 mg to 800 mg, from 700 mg to 825 mg, from 700 mg to 850 mg, from 800 mg to 825 mg, from 800 mg to 850 mg, from 800 mg to 875 mg, and from 800 mg to 900 mg) of danicopan. In one embodiment, the pharmaceutical composition is formulated as a tablet and contains 50 mg to 900 mg (e.g., 50 mg to 75 mg, 50 mg to 100 mg, 50 mg to 125 mg, 50 mg to 150 mg, 50 mg to 175 mg, 50 mg to 200 mg, 50 mg to 225 mg, 50 mg to 250 mg, 60 mg to 75 mg, 60 mg to 100 mg, from 60 mg to 125 mg, from 60 mg to 150 mg, from 60 mg to 175 mg, from 60 mg to 200 mg, 60 mg to 225 mg, from 60 mg to 250 mg, 70 mg to 75 mg, 70 mg to 100 mg, 70 mg to 125 mg, 70 mg to 150 mg, 70 mg to 175 mg, 70 mg to 200 mg, 70 mg to 225 mg, 70 mg to 250 mg, 80 mg to 100 mg, 80 mg to 125 mg, 80 mg to 150 mg, 80 mg to 175 mg, 80 mg to 200 mg, 80 mg to 225 mg, 80 mg to 250 mg, 90 mg to 100 mg, 90 mg to 125 mg, 90 mg to 150 mg, 90 mg to 175 mg, 90 mg to 200 mg, from 90 mg to 225 mg, from 90 mg to 250 mg, from 100 mg to 125 mg, from 100 mg to 150 mg, from 100 mg to 175 mg, from 100 mg to 200 mg, from 100 mg to 225 mg, from 100 mg to 250 mg, 200 mg to 225 mg, from 200 mg to 250 mg, from 200 mg to 275 mg, from 200 mg to 300 mg, from 200 mg to 325 mg, and from 200 mg to 350 mg, 300 mg to 325 mg, from 300 mg to 350 mg, from 300 mg to 375 mg, from 300 mg to 400 mg, from 300 mg to 425 mg, from 300 mg to 450 mg, from 400 mg to 425 mg, from 400 mg to 450 mg, from 400 mg to 475 mg, from 400 mg to 500 mg, from 400 mg to 525 mg, from 400 mg to 550 mg, from 500 mg to 525 mg, from 500 mg to 550 mg, from 500 mg to 575 mg, from 500 mg to 600 mg, from 500 mg to 625 mg, from 500 mg to 650 mg, from 600 mg to 625 mg, from 600 mg to 650 mg, from 600 mg to 675 mg, from 600 mg to 700 mg, from 600 mg to 725 mg, from 600 mg to 750 mg, from 700 mg to 725 mg, from 700 mg to 750 mg, from 700 mg to 775 mg, from 700 mg to 800 mg, from 700 mg to 825 mg, from 700 mg to 850 mg, from 800 mg to 825 mg, from 800 mg to 850 mg, from 800 mg to 875 mg, and from 800 mg to 900 mg) of danicopan.

### Administration

The formulations described herein can be administered to achieve any amount/and or frequency to achieve to provide an effective amount of danicopan to a host to treat a disorder modulated by complement factor D, e.g., any of the disorders described herein, which achieves the desired therapeutic result. The amount and timing of the solid dispersion formulations of danicopan administered will be dependent on the host being treated, the instructions of the supervising medical specialist, the time course of the exposure, on the manner of administration, on the pharmacokinetic properties of danicopan, and on the judgment of the prescribing physician. Thus, because of host-to-host variability, the dosages given below are a guideline and the physician can titrate doses of the compound to achieve the treatment that the physician considers appropriate for the host. In considering the degree of treatment desired, the physician can balance a variety of factors such as age and weight of the host, presence of preexisting disease, as well as presence of other diseases.

An effective amount of a formulations of danicopan as described herein can be used in an amount sufficient to (a) inhibit the progression of a disorder mediated by the complement pathway, including an inflammatory, immune, including an autoimmune, disorder or complement Factor D related disorder; (b) cause a regression of an inflammatory, immune, including an autoimmune, disorder or complement Factor D related disorder; (c) cause a cure of an inflammatory, immune, including an autoimmune, disorder or complement Factor D related disorder; or inhibit or prevent the development of an inflammatory, immune, including an autoimmune, disorder or complement Factor D related disorder. Accordingly, an effective amount of the solid dispersion formula or composition described herein will provide a sufficient amount of danicopan when administered to a patient to provide a clinical benefit.

The pharmaceutical composition may be formulated as any pharmaceutically useful form, e.g., a pill, a capsule (e.g., hard gelatin, soft gelatin, and liquid soft gelatin capsules), a tablet, or a powder. Some dosage forms, such as tablets and capsules, are subdivided into suitably sized unit doses containing appropriate quantities of the active components, e.g., an effective amount of danicopan to achieve the desired purpose.

The therapeutically effective dosage of the formulations described herein will be determined by the health care practitioner depending on the condition, size and age of the patient as well as the route of delivery. In certain embodiments the pharmaceutical composition is in a dosage form that contains from 50 mg to 900 mg, or from 100 mg to 400 mg of the danicopan. The dosage form can be administered, for example, once a day (QD), twice a day (BID), three times a day (TID), four times a day (QID), five times a day (OD), once every other day (Q2D), once every third day (Q3D), as needed, or any dosage schedule that provides treatment of a disorder described herein. In some embodiments, the dosage form is administered TID.

The formulations disclosed herein may be administered orally.

In accordance with the presently disclosed methods, an oral dosage form for administration can be in any desired form in which the formulation is stable as a solid. The solid dispersion formulations as disclosed in the present disclosure have good pharmacokinetic and pharmacodynamics properties, for instance when administered by the oral routes.

### EXAMPLES

### Example 1. Preparation of Danicopan Liquid Soft Gelatin Capsule Formulation

The active fill of the danicopan liquid soft gelatin capsule formulation was prepared at a theoretical batch size of 740 g using a high shear Silverson L4R homogenizer. Capmul MCM and D-alpha-tocopheryl PEG 1000 succinate were preheated to melt at 45° C. for weighing and transfer prior to formulation preparation. While no heat was applied to the formulation during homogenization, the temperature of the mixture increase to 64° C. from room temperature due to heat generated by the mixing blade and fill material.

A 16:2:1:1 ratio of Capmul MCM, danicopan, propylene glycol, and D-alpha-tocopheryl PEG 1000 succinate was used to prepare the danicopan liquid soft gelatin capsule formulation. In a flask, 592 g of Capmul MCM, 37 g of propylene glycol, and 37 g of D-alpha-tocopheryl PEG 1000 succinate were combined, and then 74 g of spray dried danicopan was added in approximately five portions during homogenization. The fill material was homogenized for 30 min after the complete addition of danicopan. The resulting fill material was filtered through a 0.45 micron Nylon filter to remove insoluble danicopan and foreign particulates.

To encapsulate the active fill, the fill was transferred to the 7th Generation Pilot encapsulation machine along with a separate heated tank containing the molten gel mass. The gel mass was cast into two ribbons. Both ribbons were then lubricated and passed between the rotating dies. The fill material was fed by gravity to the encapsulation pump. The pump operated by positive displacement and delivered fill material through a heated filling wedge (47° C.) between rotating dies, expanding the gel ribbons to form capsules. The dies form seals and cut capsules out from the ribbons in a continuous, hermetically sealed process. The target fill and shell weights were 1.0 and 0.606 g, respectively. After encapsulation, the soft gels were dried using two drying steps.

### Example 2. Stability Studies of Danicopan Liquid Soft Gelatin Capsule Formulation

Stability studies of the 100 mg danicopan liquid soft gelatin capsule formulation were conducted to determine the shelf life of the formulation. The clinical batches of the soft gelatin capsules were stored in thermoform blister packaging and were subjected to 40° C. and 75% relative humidity. The soft gelatin capsules were inspected monthly to monitor the API purity, and the appearance color, shape, and fill of the capsules. Large crystals of danicopan were observed in the fill of the soft gelatin capsules after 1 month under the stated conditions, indicating that the liquid soft gelatin capsule formulation has a short shelf life.

### Example 3. Preclinical Spray Dried Solid Dispersion Formulations of Danicopan

Various solid dispersion formulations of danicopan were prepared and characterized. The preferred polymers were found to be: (i) 79% (w/w) danicopan with 19% polyvinyl pyrrolidone-vinyl acetate and 2% sodium lauryl sulfate in 95:5 acetone/water; and (ii) 80% (w/w) danicopan with 20% HPMCAS-H polymer in acetone.

The following procedure was developed to prepare two spray dried solid dispersion formulations of danicopan: (i) formulation 1 (79% (w/w) danicopan with 19% polyvinyl pyrrolidone-vinyl acetate and 2% sodium lauryl sulfate in 95:5 acetone/water); and (ii) formulation 2 (80% (w/w) danicopan with 20% HPMCAS-H polymer in acetone). Table 1 summarizes the reagent quantities required to prepare the spray dried solid dispersions.

TABLE 1
Quantities of reagents for spray dried
solid dispersions of danicopan.

| Feed Properties | Formulation 1 | Formulation 2 |
|---|---|---|
| Danicopan (g)^{a} | 158.0 | 160.0 |
| Polyvinyl pyrrolidone-vinyl | 38.0 | N/A |
| acetate (copovidone) (g) | | |
| HPMCAS-H (g) | N/A | 40.0 |
| Sodium lauryl sulfate (g) | 4.0 | N/A |
| Acetone (g) | 1133.4 | 1133.4 |
| Water^{b} (g) | 64.0 | N/A |
| Total solids (g) | 200 | 200 |
| Drug load (% w/w) | 79% | 80% |
| Total liquids (g) | 1197.4 | 1133.4 |
| C feed (% w/w) | 14.3% | 15% |

^{a}The concentration of danicopan was >300 mg/mL
^{b}Water was added to dissolve sodium lauryl sulfate

A laboratory scale spray drier, SD46 (4M8-TriX Procept Spray Dryer), was used to dry the feed solution. The unit was equipped with either a two fluid nozzle (1.0 mm, from Procept) or an ultrasonic nozzle (25 kHz, from Sono-Tek). The spray drying unit was operated with nitrogen in open-loop configuration (i.e., without recirculation of the drying nitrogen). A cyclone was used to collect the dried product. Before initiating the drying, the spray dryer was stabilized (i) with drying gas, adjusting the inlet temperature (Tᵢₙ) to that estimated for the process; and then (ii) with the corresponding solvent system adjusting the feed flow (F_{feed}) to the corresponding amount of solvent expected to be sprayed. During stabilization with the solvent mixture, Tᵢₙ was adjusted to achieve the target outlet temperature (Tₒᵤₜ). After stabilization, the feed of the spray dryer was commuted from the solvent to the feed solution. F_{feed} was then readjusted to maintain Tₒᵤₜ in the target value. At the end of the spray drying, the feed was commuted to stabilization solvent mixture in order to rinse the feed line and to perform a controlled shutdown of the unit. All trials were performed under similar experimental conditions. The product collected from the cyclone was weighed and the yield calculated as the mass percentage of the wet product in relation to the total solids in the solution fed to the spray dryer.

To perform secondary drying, a vacuum tray dryer, EV52, was used to reduce the residual solvents content of the wet spray dried solid dispersion. The drying was carried out under vacuum with nitrogen sweeping for at least 24 hours. The spray dried materials were amorphous after the spray drying and secondary drying steps and did not form phase-separated systems.

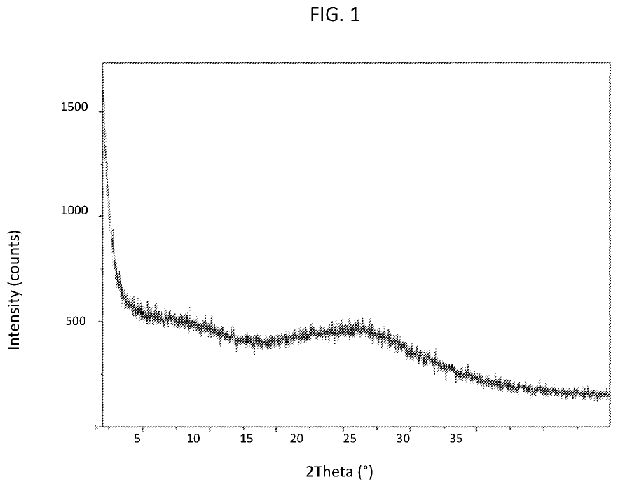

The spray dried samples were analyzed using X-ray powder diffraction (XRPD) to screen for crystalline material, and both formulations were found to be amorphous. As an example, XPRD analysis of formulation 2 exhibited an amorphous halo, with no evidence of crystallinity (FIG. 1). Percent crystallinity for any solid dispersion can be calculated from XRPD data integration of the crystalline and total observed XRPD diffraction peaks (% Crystallinity=[(area under the crystalline peaks)/(area under all peaks)]*100).

### Example 4. Preclinical Tablet Formulations and Tableting of Solid Dispersion Danicopan

Two tablet formulations using standard oral dosage form excipients were tested for each one of the danicopan solid dispersion formulations, rendering four total tablet formulations of danicopan. The target tablet strength was 250 mg of danicopan in total tablet size of 600 mg. The two tablet formulations differed in the filler used (Tablettose 80 for Formulation A, and Avicel PH102 for Formulation B). Three batches of tablets were produced at low, medium, and high tablet hardness for each one of the four formulations. The prepared blends are described in Table 2.

TABLE 2
Preclinical tablet formulations of solid dispersions of danicopan.

| | 250 mg danicopan | | | |
|---|---|---|---|---|
| Dose | mg/tablet | % w/w/tablet | mg/tablet | % w/w/tablet |

Formulation A

| | | | | |
|---|---|---|---|---|
| Formulation 1 | 316.5 | 52.7 | N/A | N/A |
| Formulation 2 | N/A | N/A | 312.5 | 52.1 |
| Croscarmellose | 30.0 | 5.0 | 30 | 5.0 |
| sodium | | | | |
| Magnesium | 3.0 | 0.5 | 3 | 0.5 |
| stearate | | | | |
| Aerosil | 6.0 | 1.0 | 6 | 1.0 |
| Tablettose 80 | 244.5 | 40.8 | 248.5 | 41.4 |
| Total | 600 | 100 | 600 | 100 |

Formulation B

| | | | | |
|---|---|---|---|---|
| Formulation 1 | 316.5 | 52.7 | N/A | N/A |
| Formulation 2 | N/A | N/A | 312.5 | 52.1 |
| Croscarmellose | 30.0 | 5.0 | 30 | 5.0 |
| sodium | | | | |
| Magnesium | 3.0 | 0.5 | 3 | 0.5 |
| stearate | | | | |
| Aerosil | 6.0 | 1.0 | 6 | 1.0 |
| Avicel PH102 | 244.5 | 40.8 | 248.5 | 41.4 |
| Total | 600 | 100 | 600 | 100 |

The tablets were further characterized in terms of friability, and disintegration (Tables 3 and 4). Hardness was tested for values of approximately 100 N, 150 N and 200 N to render low, medium, and high hardness 600 mg tablets.

TABLE 3
Tablet properties for Formulation A.

| Formulation | 1A | 1A | 1A | 2A | 2A | 2A |
|---|---|---|---|---|---|---|
| Hardness (N) | 180-220 | 130-170 | 80-120 | 180-220 | 130-170 | 80-120 |
| Tablet Weight (g) | 597.3 ± 10 | 601 ± 10 | 597.0 ± 11 | 593 ± 3 | 596 ± 7 | 602 ± 4 |
| Thickness (mm) | 5.3 ± 0.1 | 5.9 ± 0.0 | 6.4 ± 0.0 | 5.5 ± 0.0 | 5.8 ± 0.0 | 6.2 ± 0.0 |
| Wall height (mm) | 2.6 ± 0.2 | 3.2 ± 0.3 | 3.6 ± 0.1 | 2.6 ± 0.1 | 3.1 ± 0.0 | 3.4 ± 0.0 |
| Friability (%) | 0.21 | 0.26 | 0.42 | 0.07 | 0.03 | 0.14 |
| Disintegration | 4:54 | 0:27 | 0:23 | 2:29 | 1:59 | 1:17 |
| (mm:ss) | | | | | | |

TABLE 4
Tablet properties for formulation B.

| Formulation | 1B | 1B | 1B | 2B | 2B | 2B |
|---|---|---|---|---|---|---|
| Hardness (N) | 180-220 | 130-170 | 80-120 | 180-220 | 130-170 | 80-120 |
| Tablet Weight (g) | 602 ± 9 | 592 ± 11 | 601 ± 18 | 597 ± 8 | 597 ± 4 | 598 ± 3 |
| Thickness (mm) | 6.3 ± 0.0 | 6.4 ± 0.0 | 7.5 ± 0.2 | 6.2 ± 0.1 | 6.6 ± 0.0 | 7.1 ± 0.0 |
| Wall height (mm) | 3.5 ± 0.1 | 3.9 ± 0.0 | 4.6 ± 0.2 | 3.4 ± 0.0 | 3.7 ± 0.0 | 4.3 ± 0.0 |
| Friability (%) | 0.07 | 0.24 | 0.56 | 0.05 | 0.22 | 0.08 |
| Disintegration | 0:26 | 0:23 | 0:17 | 1:33 | 0:41 | 0:29 |
| (mm:ss) | | | | | | |

Friability (loss weight) results were acceptable for medium and high hardness tablets; thus, these formulations allow working within the hardness range of 150-200 N. The low hardness tablets tend to break during the friability test and therefore were considered inadequate for manufacturing. Lastly, all formulations showed low disintegration times, less than five minutes even for the highest hardness tablets. Tablet disintegration was consistently faster in the Formulation B samples.

### Example 5: Dissolution Kinetics Assay of Preclinical Solid Dispersion Formulations of Danicopan

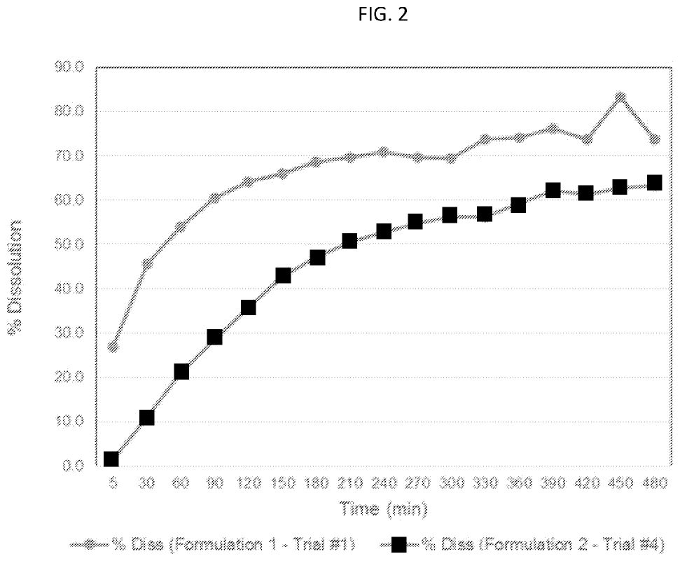

A dissolution kinetics assay was performed to determine the dissolution kinetics of formulations 1 and 2. Secondary-dried samples of formulations 1 and 2 were dissolved in aqueous 0.1 N hydrochloric acid at 37° C. with 75 rpm stirring, and the dissolution of danicopan was monitored over time using HPLC. Formulation 1 exhibited faster dissolution kinetics compared to formulation 2, where it took approximately 40 min to dissolve at least 50% (w/w) of formulation 1, and approximately 210 min to dissolve at least 50% (w/w) of formulation 2 (FIG. 2).

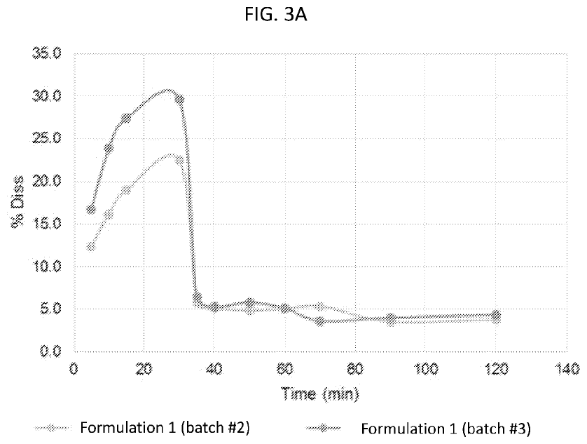

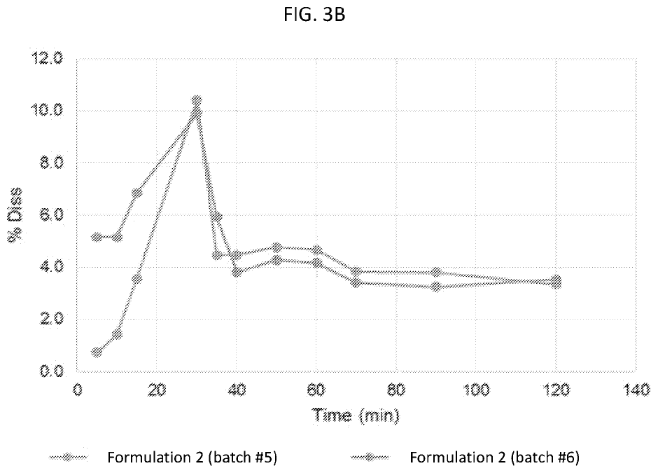

A pH shift dissolution assay was performed to monitor the dissolution kinetics of formulations 1 and 2 when the pH of the dissolution medium increases from pH 1 to pH 4-5 (FIGS. 3A and 3B). Secondary-dried samples of formulations 1 and 2 were dissolved in aqueous 0.1 N hydrochloric acid at 37° C. with 75 rpm stirring, and the dissolution of danicopan was monitored over time using HPLC. After 30 min, the final pH of the dissolution medium was increased to pH 4-5 by the addition of 10 mM phosphate buffer and 1 M aqueous NaOH to the dissolution medium. After 30 min, approximately 23-30% of formulation 1 was dissolved, while approximately 10-11% of formulation 2 was dissolved. After the pH was adjusted to pH 4-5, the dissolution of formulation 1 remained at 5% over time, while the dissolution of formulation 2 remained at 4-4.5% over time. The results indicate that formulation 1 dissolves faster than formulation 2, even when the pH of the solution increases.

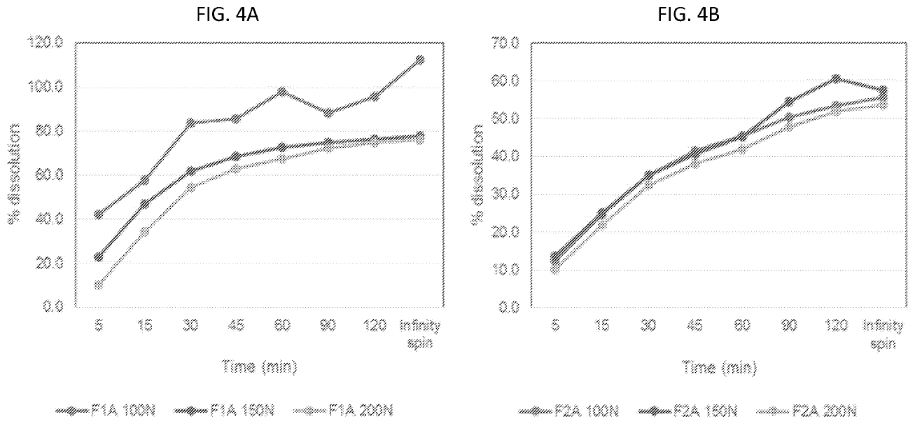

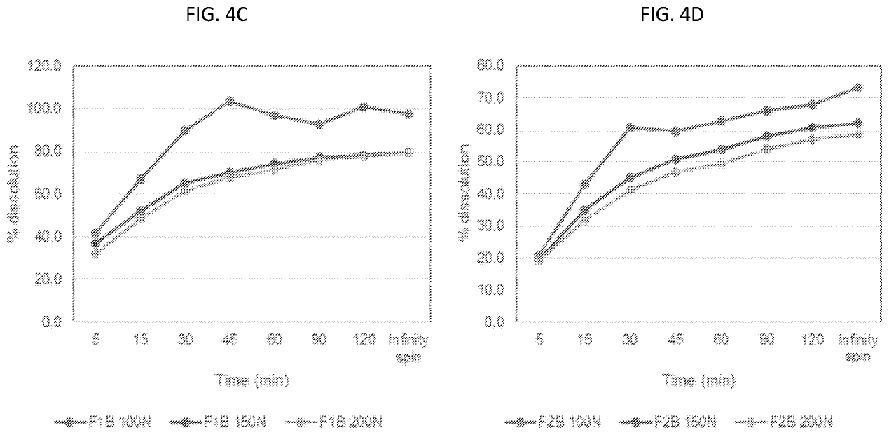

The dissolution kinetics of the tableted forms of formulation 1 (A and B) and formulation 2 (A and B) were also determined using a dissolution kinetics assay. The tablets were dissolved in aqueous 0.1 N hydrochloric acid at 37° C. with 50 rpm stirring, and the dissolution of danicopan was monitored using HPLC. The tableted forms of formulation 1 exhibited faster dissolution kinetics compared to tableted forms of formulation 2 (FIG. 4A-D). For example, after 2 hours, the dissolution testing showed a drug dissolution of approximately 80% and 60% in formulation 1 (A and B) and formulation 2 (A and B), respectively.

Although formulation 1 showed a faster drug dissolution than formulation 2, based on preclinical studies, formulation 2 was selected for subsequent scale-up campaigns and GMP manufacturing.

### Example 6: Supersaturation Profiles of Danicopan/HPMCAS Solid Dispersions in Simulated Gastric Fluid (SGF) or Simulated Intestinal Fluid (SIF)

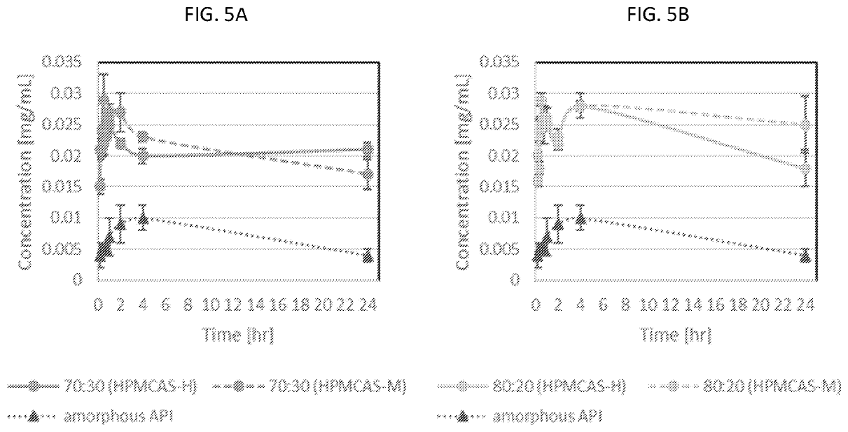

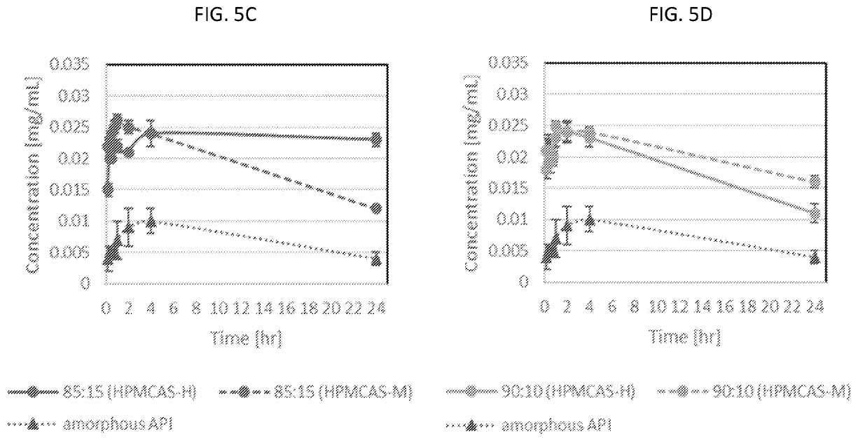

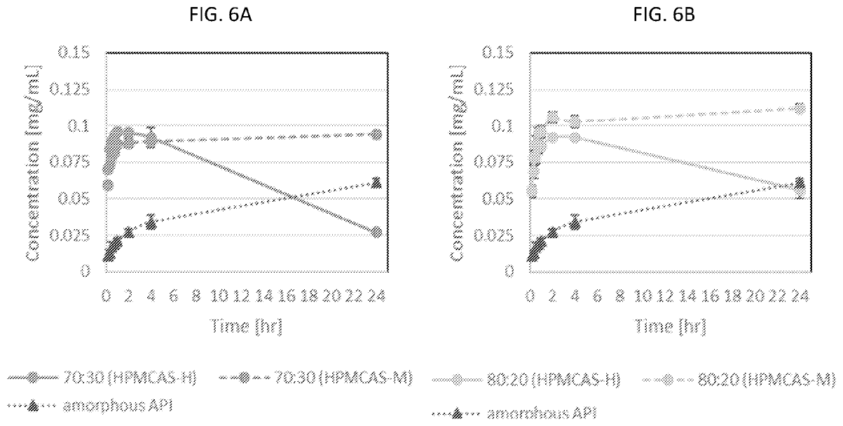

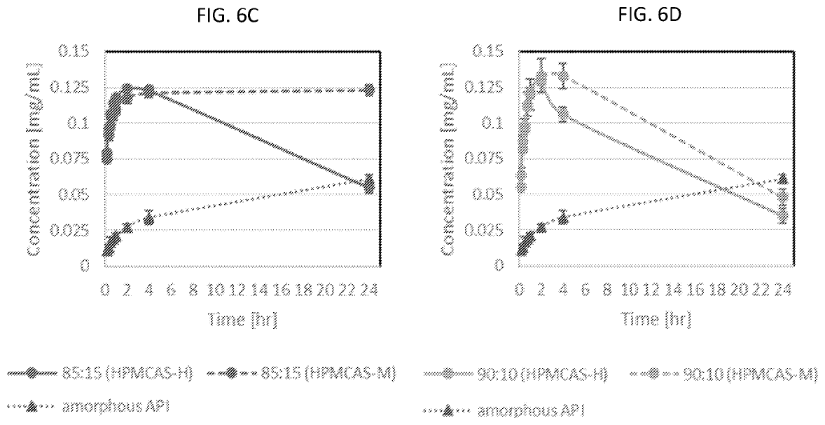

Non-sink in vitro solubility screening was performed to determine the effect of HPMCAS grade and danicopan loading on the extent and duration of supersaturation and solubility of the solid dispersions. Samples were tested in 40 mL simulated gastric fluid (SGF) or simulated intestinal fluid (SIF) at 37° C. for 24 hours. Samples were taken at 10, 20, 30, 45 minutes and at 1, 2, 4 and 24 hours and analyzed using HPLC. Graphs showing the in vitro dissolution behavior of each solid dispersion composition in SIF are shown in FIG. 5A-D and the in vitro dissolution results in SGF are shown in FIG. 6A-D.

FIG. 5A-D and FIG. 6A-D show that higher concentrations of danicopan are reached in SGF than in SIF, which is consistent with the higher solubility of the danicopan in acidic media. In SGF, the solid dispersion made from the M grade polymer generally seemed to sustain better than H grade (for the same danicopan/HPMCAS ratio). Since the solid dispersion is presumably only in the stomach for a relatively short time (<2 hours), this may not be relevant in vivo. In SIF, after 4 hours, equivalent performance is seen between solid dispersions made with H and M grade polymer for each danicopan/HPMCAS ratio. Given that most of the absorption would be expected to occur in this time frame, this is probably most relevant. After 24 hours in SIF, the higher danicopan loaded solid dispersions showed a significant drop in the concentration of danicopan in solution, although it was still above the amorphous solubility in all cases, indicating that bioavailability should be enhanced relative to the amorphous danicopan alone.

### Example 7: Tablet Formulation of 80:20 Danicopan/HPMCAS-H Solid Dispersion for Large Scale Production

The tablet formulation of the 80:20 danicopan/HPMCAS-H spray dried solid dispersion was optimized for large scale production. The 100 mg formulation used a 1:1 intra-granular ratio of spray dried solid dispersion to filler (combination of MCC and lactose) and had a total tablet size of 400 mg. Both intra- and extra-granular filler were 50:50 MCC:lactose. The 100 mg tablet formulation composition is shown in Table 5:

TABLE 5
100 mg tablet formulation of 80:20 danicopan/HPMCAS-H
SDD in a total tablet size of 400 mg.

| Ingredients | 100 mg tablet formulation | | |
|---|---|---|---|
| Intra-granular | mg/tab | % Intragranular | % tablet |
| 80:20 danicopan/HPMCAS-H | 125 | 46.47% | 31.25% |
| SDD | | | |
| Lactose (Granulose 200) | 63 | 23.42 | 15.75% |
| Microcrystalline cellulose | 62 | 23.05% | 15.50% |
| PH101 | | | |
| Croscarmellose sodium | 10 | 3.72% | 2.50% |
| Sodium lauryl sulphate | 8 | 2.97% | 2.00% |
| Magnesium stearate | 1 | 0.37% | 0.25% |
| Intra-granular sub-total | 269 | | 67.25% |
| Extra-granular | | | |
| Lactose (Tablettose 80) | | | 14.50% |
| Microcrystalline cellulose | | | 14.50% |
| PH102 | | | |
| Croscarmellose sodium | | | 2.50% |
| Colloidal silicon dioxide | | | 1.00% |
| Magnesium stearate | | | 0.25% |
| Tablet total | 400 | | 100.0% |

### Example 8: Pharmacokinetics of Danicopan Solid Dispersion Formulations in Canines

Five danicopan SDD compositions were tested in canines to determine the pharmacokinetics (PK) the formulations: (1) 70:30 (danicopan/HPMCAS-H), (2) 80:20 (danicopan/HPMCAS-H), (3) 85:15 (danicopan/HPMCAS-H); (4) 80:20 (danicopan/HPMCAS-M), and (5) 85:15 (danicopan/HPMCAS-M).

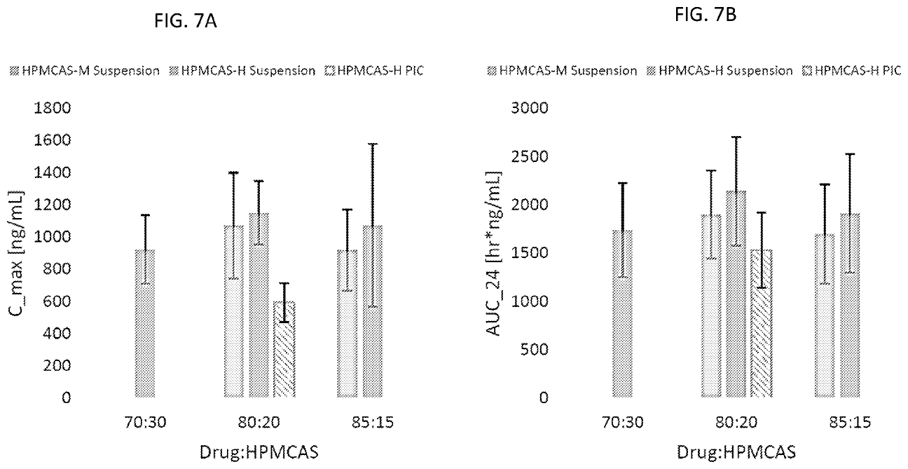

The danicopan solid dispersion formulations were administered in a cross-over design to five dogs in 6 dose events with a washout period of ~7 days between successive doses, except between dose events 2 and 3 which were separated by ~3 weeks. In dose event 1, canines were administered the solid dispersion as a 50 mg oral capsule. For dose events 2-6, solid dispersions formulated as suspensions in 0.5% HPMC (w/w) and 0.1% Tween (w/V) were administered as oral gavages at a dose level of 50 mg/canine and a dose volume of 10 mL/dog. All canines were fasted overnight and pretreated with 6 μg/kg pentagastrin 20 minutes prior to dosing. A summary of PK results is shown in Table 6 and graphically in FIGS. 7A and 7B. Results show no significant differences in Cₘₐₓ or AUC mean values or variability for either different danicopan loadings or HPMCAS polymer grades for SDDs dosed as a suspension. Danicopan SDD dosed as a powder-in-capsule (PIC) showed approximately ½ the Cmax and ¾ the AUC when compared to dosing as a suspension. These data support that the current composition of the danicopan SDD is in a robust formulation space.

TABLE 6
Pharmacokinetics of danicopan solid dispersion formulations in canines

| | | | Mean (SD) | | |
|---|---|---|---|---|---|
| SDD | | | | Cmax | AUC₀₋₂₄ |
| composition | Formulation | Dose event | Tmax (hr) | (ng/mL) | (h*ng/mL) |
| 70:30 H grade | Suspension | 6 | 0.5 (0.5-1) | 922 (214) | 1740 (488) |
| 80:20 H grade | Suspension | 3 | 0.5 (0.5-0.5) | 1150 (198) | 2140 (563) |
| 80:20 H grade | PIC | 1 | 1 (1-1) | 592 (121) | 1530 (389) |
| 85:15 H grade | Suspension | 5 | 0.5 (0.5-0.5) | 1070 (507) | 1910 (616) |
| 80:20 M grade | Suspension | 2 | 0.5 (0.5-0.5) | 1070 (329) | 1900 (457) |
| 85:15 M grade | Suspension | 4 | 0.5 (0.5-0.5) | 917 (252) | 1700 (512) |

### Example 9. Additional Spray Dried Solid Dispersion Formulations of Danicopan

The following spray dried solid dispersion formulations of danicopan were developed for screening purposes (Table 7). The solvent system was selected as 80:20 dichloromethane:methanol (w/w) based on the solubility screen and in order to prepare all the solid dispersions under the same spray conditions.

TABLE 7
Additional spray dried solid dispersion formulations of danicopan

| | | | 25/75 | | | 80/20 | 25/75 |
|---|---|---|---|---|---|---|---|
| | 25/75 | 25/75 | danicopan/ | 25/75 | 25/75 | danicopa | danicopan/ |
| | danicopan/ | danicopan/HP | Eudragit L- | danicopan/ | danicopan/PVP | n/HPMCA | HPMCAS- |
| Component | HPMCAS-M | MC E3 | 100 | PVP VA64 | K30 | S-H | H |
| Danicopan | 1.25 | 1.25 | 1.25 | 1.25 | 1.25 | 4.0 | 1.25 |
| (g) | | | | | | | |
| Polymer (g) | 3.75 | 3.75 | 3.75 | 3.75 | 3.75 | 1.0 | 3.75 |
| Solvent | 95.00 | 95.00 | 95.00 | 95.00 | 95.00 | 95.00 | 95.00 |
| system: | | | | | | | |
| 80/20 | | | | | | | |
| DCM/MeOH | | | | | | | |

The above spray dried solid dispersions were characterized by XRPD, modulated differential scanning calorimetry (mDSC), thermogravimetric analysis (TGA), scanning electron microscopy (SEM), and polarized light microscopy (PLM), summarized in Table 8.

TABLE 8
Characterization of additional spray dried solid dispersions using XRPD, modulated
differential scanning calorimetry (mDSC), thermogravimetric analysis (TGA),
scanning electron microscopy (SEM), and polarized light microscopy (PLM)

| | | | | TGA | Microscopy |
|---|---|---|---|---|---|
| | | SDD mDSC | Polymer mDSC | (% wt change | morphology |
| Solid dispersion | XRPD | (Tg, ° C.) | (Tg, ° C.) | to 125° C.) | (SEM/PLM) |
| 25/75 | Amorphous | 111.16 | 120 | 2.49% | Amorphous |
| danicopan/HPMCAS-M | | | | | |
| 25/75 | Amorphous | 111.50 | 126 | 2.21% | Amorphous |
| danicopan/HPMCAS-H | | | | | |
| 25/75 danicopan/HPMC | Amorphous | 121.32 | 145 | 4.48% | Amorphous |
| E3 | | | | | |
| 25/75 danicopan/PVP | Amorphous | 119.43 | 108 | 4.94% | Amorphous |
| VA64 | | | | | |
| 25/75 danicopan/PVP | Amorphous | 156.83 | 160 | 7.03% | Amorphous |
| K30 | | | | | |
| 25/75 | Amorphous | 181.092 | 127 | 5.108% | Amorphous |
| danicopan/Eudragit L- | | | | | |
| 100 | | | | | |
| 80/20 | Amorphous | 121.00 | 126 | 2.15% | Amorphous |
| danicopan/HPMCAS-H | | | | | |

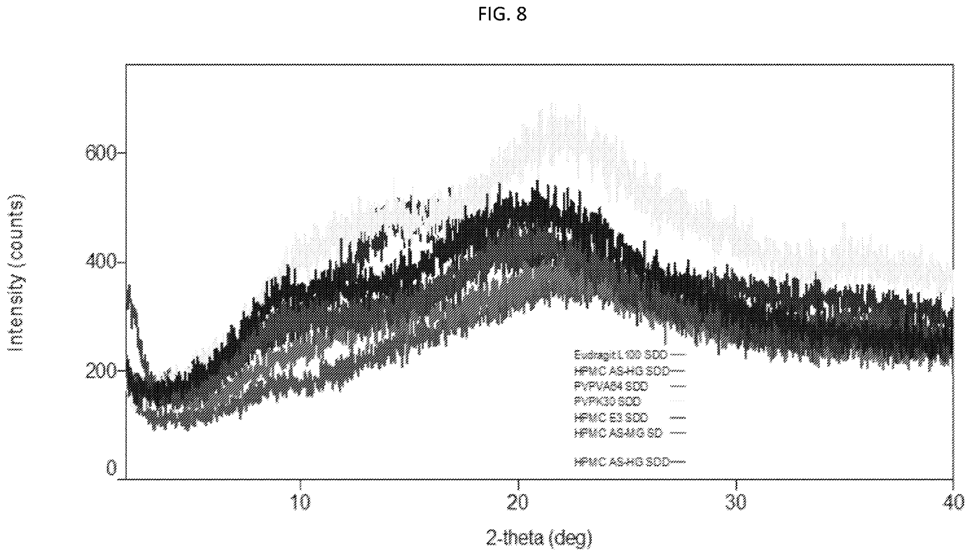

Physical characterization test results by XRPD, mDSC, and microscopy indicated that all the spray dried solid dispersions produced were completely amorphous. The XRPD diffractogram for each solid dispersion was clear of sharp crystalline peaks as can be seen in FIG. 8. All mDSC thermograms indicated a single Tg (single phase system) for each of the polymer systems indicating an amorphous dispersion. However, the Eudragit L100 SDD showed a Tg close to 180° C. which is greater than the 173-176° C. onset degradation temperature for the polymer and is greater than the Tg of the polymer which was measured at 127° C. The rerun results confirmed that the Tg of the polymer system was higher than expected and was around 180° C.

TGA showed most polymer systems contain 2-4% residual solvent/moisture which is a normal range for prototype solid dispersion formulations, except for Eudragit L 100 and PVP K30 SDD's which had >5% residual solvent which is on the high end. The initial microscopy characterization data suggested that all of the solid dispersions are in amorphous state with no indication of detectable crystallinity. The DSC data showed that they all are single phase systems with one Tg and no indication of melt. All of the solid dispersion particles are shriveled spheres and under polarized light microscopy exhibited no birefringence, indicating lack of crystallinity. The particle size of the solid dispersions ranged from anywhere between 2 to 25 μm.

The μDISS Profiler™ instrument from Pion, Inc was used to quantify concentrations during kinetic solubility experiments. The unit consists of six photodiode array (PDA) spectrophotometers, each with its own dedicated fiber optic dip probe, center-positioned in the glass vial holding 10 ml of media. The concentration measurements are performed directly in the assay media, with processed results plotted in “real time.”

Probes with 5-mm and 2-mm (for solid dispersions comprising of polymers: HPMCAS-M, Eudragit L100 and HPMCAS-H) path length tips were selected for quantification of danicopan in solid dispersion formulations and 20-mm path length tips for the API. The developed calibration curves were used for quantification of danicopan in the samples during kinetic solubility experiments at each time point. The above specified path length tips were selected for detecting concentrations of the danicopan in simulated gastric fluid (SGF) and fasted state simulated intestinal fluid (FaSSIF) buffers.

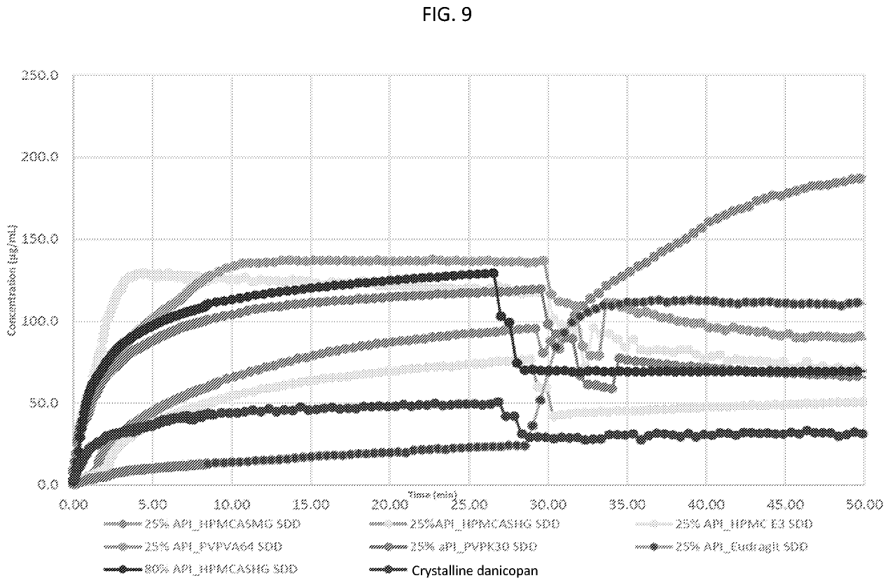

About 42 mg of solid dispersion materials, equivalent to 10 mg of danicopan were weighed into a 20-mL glass vial. The vials are then transferred to the instrument for analysis. A clean stir bar was added to the vial with sample. 16 mL of SGF buffer is transferred to the vials before beginning the experiment to achieve an upper limit of ~650 μg/mL. The stirring was maintained at 220 RPM and the temperature of the medium at 37° C. Kinetic solubility data was collected for 30 minutes in SGF media. The data showed that the solid dispersion has much higher solubility in the media when compared to danicopan (FIG. 9). The maximum solubility in most of the solid dispersion materials was at least 2 times higher than the crystalline danicopan. The HPMCAS-M, HPMCAS-H, and Eudragit solid dispersions did not show higher release at this low pH since these are acidic in nature and tends to ionize and release after pH 5.5 at higher pH.

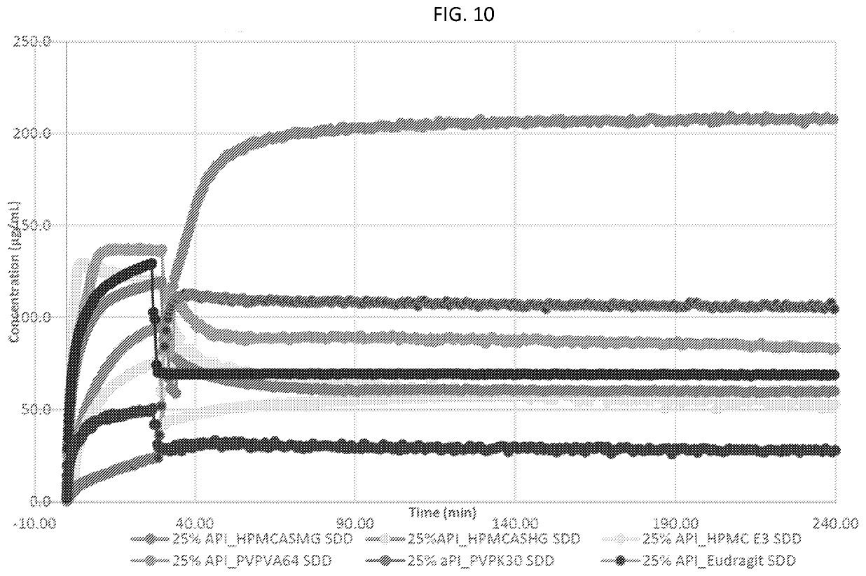

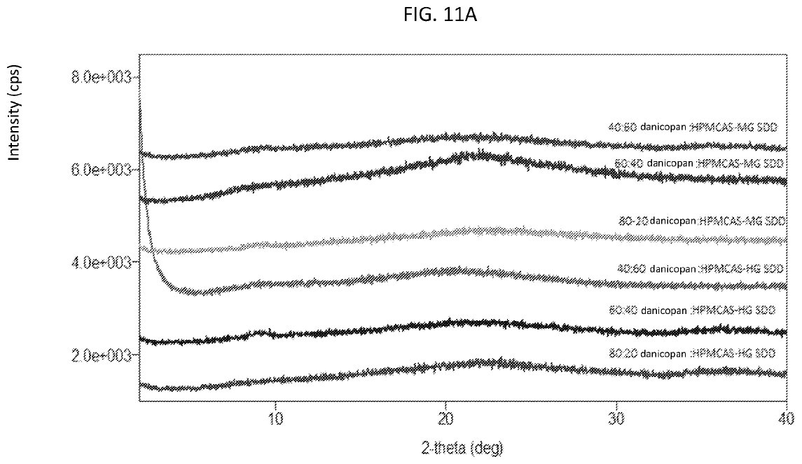

At the 30-minute interval, the media was converted to FaSSIF 6.5 using in house media conversion protocol. The final volume in the vials is increased to 20 mL from 16 mL (500 μg/mL). The resulting samples were then analyzed using the μDiss Profiler for ~18 hours in FaSSIF. All the solid dispersion formulations exhibited 2 to 5 times higher equilibrium solubility when compared to the crystalline danicopan at 4 hours. The PVP VA64 solid dispersion showed higher spring and parachute effect, staying in supersaturated state around ~85 μg/mL, followed by 80:20 danicopan/HPMCAS-H solid dispersion (control SDD) stabilizing at ~65 μg/mL, respectively (FIG. 10). There were spectral shifts observed in HPMCAS-M, HPMCAS-H and Eudragit L100 solid dispersions (25% danicopan loading), which influenced the data.

The experiments were repeated for the solid dispersions which showed issues by reducing the upper limit to 0.25 mg/ml of danicopan in FaSSIF and changing the path length tips to 2 mm to enable better detection, preventing the scattering of light from excess solids in the vials. Although there was improvement in the quality of data collected under new experimental settings, there was still some small spectral shifts observed in HPMCAS-M and Eudragit L100 solid dispersions. These shifts are not huge and within the wavelength region selected for quantification.

These formulations were assessed based on their equilibrium solubility and maximum concentration observed during the assays. The concentration profiles collected in SGF and FaSSIF are shown in FIG. 9 and FIG. 10. All experiments were conducted at N=2 replicates for each solid dispersion and the results were summarized in Table 9 below. The data suggested that the HPMCAS-M solid dispersion outperformed other solid dispersion systems in terms of achieving higher solubility and staying in supersaturated state at ~200 μg/mL. The Eudragit solid dispersion reached ~120 μg/mL in FaSSIF and stayed supersaturated for more than 4 hours. Most importantly, all of the solid dispersion formulations had improved Cₘₐₓ and kinetic solubility profiles compared to crystalline danicopan (Table 9). The PVP VA64 solid dispersion and HPMCAS-H solid dispersion also performed better than the other solid dispersion systems by achieving ~85 μg/mL and ~65 μg/mL, respectively. The rationale behind considering the 4 hour time point is to mimic the transit times in an in-vivo environment, where drugs are subjected to metabolism and excretion after this time period as they pass through GI tract.

TABLE 9
Summary of kinetic solubility studies of danicopan solid dispersions in fasted state
simulated intestinal fluid (FaSSIF).

| | *25/75 | *25/75 | 25/75 | 25/75 | 25/75 | ** 25/75 | |
|---|---|---|---|---|---|---|---|
| | danicopan/ | danicopan/ | danicopan/ | danicopan/ | danicopan/ | danicopan/ | Crystalline |
| | HPMCAS-M | HPMCAS-H | HPMC E3 | PVP VA64 | PVP K30 | Eudragit L-100 | danicopan |
| Simulated | 209.11 ± | 75.40 ± 1.0 | 129.51 ± | 137.07 ± | 118.92 ± | 112.4 ± 0.4 | 49.71 ± 1.14 |
| Cₘₐₓ* | 0.28 μg/mL | μg/mL | 1.69 μg/mL | 2.14 μg/mL | 2.69 μg/mL | μg/mL | μg/mL |
| SGF (30 | 92.31 ± 4.37 | 74.79 ± 1.41 | 119.11± | 136.48 ± | 118.32 ± | 23.27 ± 0.76 | 49.03 ± 1.67 |
| min) | μg/mL | μg/mL | 11.20 μg/mL | 2.98 μg/mL | 2.97 μg/mL | μg/mL | μg/mL |
| FaSSIF (4 h) | 207.97 ± | 60.07 ± 0.37 | 52.69 ± 2.20 | 83.13 ± 1.52 | 59.85 ± 0.44 | 105.94 ± | 27.34 ± 0.36 |
| | 0.52 μg/mL | μg/mL | μg/mL | μg/mL | μg/mL | 0.81 μg/mL | μg/mL |
| FaSSIF (Eq) | 199.22 ± | 69.04 ± 0.32 | 48.61 ± 0.06 | 74.15 ± 0.65 | 58.84 ± 0.32 | 105.24± | 15.34 ± 8.63 |
| | 0.87 μg/mL | μg/mL | μg/mL | μg/mL | μg/mL | 0.72 μg/mL | μg/mL |

*The simulated Cmax corresponds to the maximum concentration in the kinetical solubility profile.
**The data may be affected by a slight spectral shift seen in the solid dispersion samples during the assay.

Next, various loadings of danicopan (40%, 60%, and 80% danicopan) were tested in the solid dispersion systems to optimize the loading percentage of danicopan in the solid dispersion formulations. Table 10 describes the compositions of the various danicopan loadings that were used for each solid dispersion system.

TABLE 10
Descriptions of solid dispersions at different danicopan loadings.

| | | | Weight of |
|---|---|---|---|
| | | Danicopan | danicopan |
| Polymer | Solid dispersion description | loading | (g) |
| HPMCAS-M | 40:60 danicopan/HPMCAS-M | 40% | 6.08 |
| | 60:40 danicopan/HPMCAS-M | 60% | 4.34 |
| | 80:20 danicopan/HPMCAS-M | 80% | 3.33 |
| HPMCAS-H | 40:60 danicopan/HPMCAS-H | 40% | 6.28 |
| | 60:40 danicopan/HPMCAS-H | 60% | 4.25 |
| | 80:20 danicopan/HPMCAS-H | 80% | 3.24 |
| PVP VA64 | 40:60 danicopan/PVP VA64 | 40% | 6.24 |
| | 60:40 danicopan/PVP VA64 | 60% | 4.18 |
| | 80:20 danicopan/PVP VA64 | 80% | 3.19 |
| Eudragit | 40:60 danicopan/Eudragit L100 | 40% | 5.64 |
| L100 | 60:40 danicopan/Eudragit L100 | 60% | 3.78 |
| | 80:20 danicopan/Eudragit L100 | 80% | 3.04 |

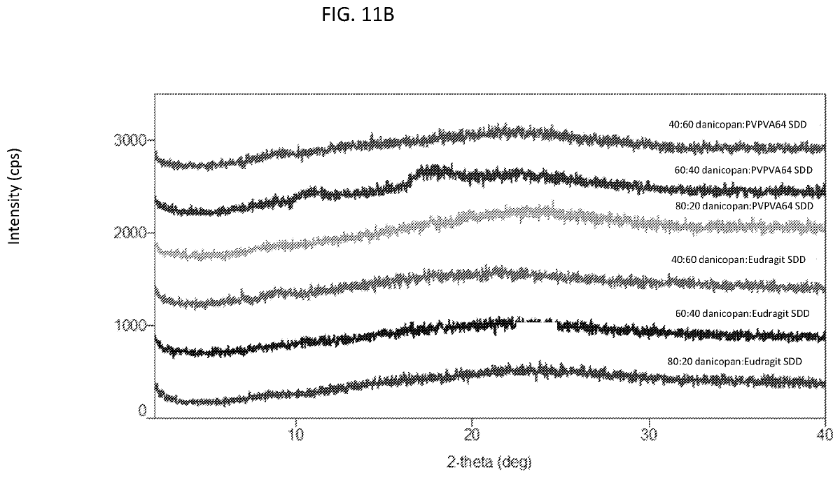

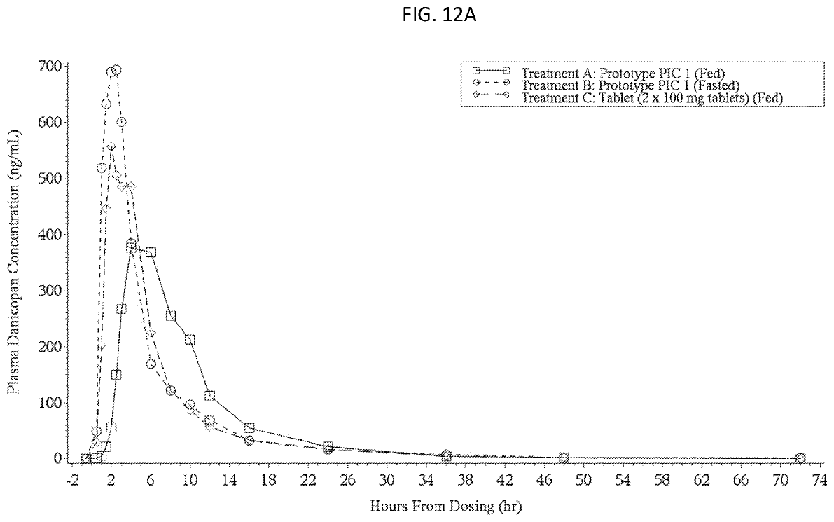

Physical characterization test results by XRPD indicated that all the spray dried dispersions described in Table 10 were completely amorphous. The XRPD diffractograms for each spray dried solid dispersion was clear of sharp crystalline peaks as can be seen in FIGS. 11A and 11B.

The kinetic solubility experiments were conducted using the same instrumentation and protocol described above in this Example. The 2 mm path length tips were used for quantification of danicopan in both SGF and FaSSIF respectively.

Required amount of solid dispersion materials, equivalent to 5 mg of danicopan were weighed into a 20-mL glass vial. The vials are then transferred to the instrument for analysis. A clean stir bar was added to the vial with sample. 16 mL of SGF buffer is transferred to the vials before beginning the experiment to achieve an upper limit of ~300 μg/mL. The stirring was maintained at 220 RPM and the temperature of the medium at 37° C. Kinetic solubility data was collected for 30 minutes in SGF media. At the 30-minute interval, the media was converted to FaSSIF pH 6.5 using in house media conversion protocol. The final volume in the vials is increased to 20 mL from 16 mL (250 μg/mL). The resulting samples were then analyzed using the μDiss Profiler for ~18 hours in FaSSIF.

All of the solid dispersion formulations exhibited 2 to 5 times higher equilibrium solubility when compared to the crystalline danicopan at 4 hours. The PVP VA64 solid dispersion showed higher spring and parachute effect, the three solid dispersions at different loading reached about ~190 g/mL in SGF but dropped to lower concentration after converting to FaSSIF. In SGF media, the 60/40 danicopan/PVP VA64 solid dispersion had attained higher equilibrium solubility when compared to the other two PVP VA64 solid dispersions at 40% and 80% danicopan loading, respectively. The 80/20 danicopan/HPMCAS-H solid dispersion also showed ability to reach higher solubility of ~100 μg/mL in SGF when compared to other solid dispersions with enteric polymers. In FaSSIF media, all the HPMCAS-M solid dispersions performed better by reaching ~100 g/mL and staying supersaturated at that concentration. Although the 40/60 danicopan/Eudragit L100 solid dispersion reached ~120 μg/mL, the data is highly affected by the spectral shift and hence may not be reliable. The kinetic solubility results are summarized in Table 11. The data suggested that the HPMCAS-M solid dispersion out-performed the other solid dispersion systems in terms of achieving higher solubility and staying in a supersaturated state at ~100 g/mL. XRPD was performed on residual wet solids after the kinetic solubility tests and the results showed that the samples did not show any signs of crystallinity in detectable levels.

TABLE 11
Summary of kinetic solubility studies of solid dispersions at different danicopan
loadings in fasted state simulated intestinal fluid (FaSSIF).

| | | HPMCAS-M SDD | | | HPMCAS-H SDD | | | PVP VA64 SDD | | | Eudragit SDD | | |
|---|---|---|---|---|---|---|---|---|---|---|---|---|---|
| Time | | 40% | 60% | 80% | 40% | 60% | 80% | 40% | 60% | 80% | 40% | 60% | 80% |
| point | Value | API | API | API | API | API | API | API | API | API | API | API | API |
| 0.5 h | Average | 59.79 ± | 64.77 ± | 68.15 ± | 81.87 ± | 79.82 ± | 105.94 ± | 181.29 ± | 184.48 ± | 172.36 ± | 35.35 ± | 31.31 ± 5.68 | 53.79 ± |
| | Conc | 3.69 | 5.16 | 7.19 | 8.23 | 8.05 | 12.16 | 13.06 | 14.13 | 19.52 | 5.68 | | 3.43 |
| | (μg/mL) | | | | | | | | | | | | |
| 4 h | Average | 104.97 ± | 97.24 ± | 96.97 ± | 61.97 ± | 60.19 ± | 67.88 ± | 72.11 ± | 92.53 ± | 62.73 ± | 127.34 ± | 55.59 ± 0.70 | 60.06 ± |
| | Conc | 0.28 | 0.24 | 0.23 | 0.19 | 0.20 | 0.16 | 0.37 | 0.42 | 0.15 | 0.69 | | 0.23 |
| | (μg/mL) | | | | | | | | | | | | |
| −16 h | Average | 98.50 ± | 90.51 ± | 90.74 ± | 65.23 ± | 59.62 ± | 67.77 ± | 83.44 ± | 76.55 ± | 61.14 ± | 126.72 ± | 78.79 ± 13.40 | 59.85 ± |
| | Conc | 0.65 | 0.54 | 0.47 | 0.30 | 0.22 | 0.12 | 28.37 | 0.35 | 0.12 | 0.79 | | 0.26 |
| | (μg/mL) | | | | | | | | | | | | |
| Simulated | Average | 105.75 ± | 97.71 ± | 99.43 ± | 88.16 ± | 86.18 ± | 114.21 ± | 188.50 ± | 192.62 ± | 183.27 ± | 128.53 ± | 93.45 ± 5.36 | 61.09 ± |
| Cₘₐₓª | Conc | 0.17 | 0.09 | 0.12 | 1.31 | 0.98 | 0.88 | 0.65° C. | 0.32 | 0.24 | 0.14 | | 0.07 |
| | (μg/mL) | | | | | | | | | | | | |

^{a}The simulated Cmax corresponds to the maximum concentration in the kinetical solubility profile.

### Example 10: Pharmacokinetics of Danicopan Solid Dispersions in Healthy Adult Humans

The pharmacokinetics of three danicopan solid dispersions were studied in healthy human volunteers. This trial was conducted in accordance with the ethical principles of Good Clinical Practice (GCP), according to the International Council for Harmonisation (ICH) Harmonized Tripartite Guideline. This was a 2-part, open-label, randomized, single-dose, 3-sequence, 3-period crossover, relative bioavailability (BA), and food-effect study in healthy adult participants. The following three danicopan solid dispersions were used in this study: (1) 60/40 danicopan/PVP VA64 solid dispersion as a 200 mg PIC (labeled as “PIC 1” throughout), (2) 25/75 danicopan/HPMCAS-M solid dispersion as a 200 mg PIC (labeled as “PIC 2” throughout), and (3) 80/20 danicopan/HPMCAS-H solid dispersion in a 100 mg tablet (labeled as “tablet” throughout).

### Part 1 of Study

On Day 1 of each period, participants received a single oral dose of danicopan as the prototype PIC 1 formulation under fed conditions (Treatment A), the prototype PIC 1 formulation under fasting conditions (Treatment B), or the tablet formulation under fed conditions (Treatment C). Participants were randomized to 1 of 3 sequences, in a ratio of 1:1:1. All participants received Treatments A, B, and C according to the treatment sequence which the participant was randomized to: ABC, BCA, or CAB. PK blood samples were collected for danicopan predose and for 72 hours following each danicopan administration.

### Part 2 of Study

On Day 1 of each period, participants received a single oral dose of danicopan as the prototype PIC 2 formulation under fed conditions (Treatment D), the prototype PIC 2 formulation under fasting conditions (Treatment E), or the tablet formulation under fed conditions (Treatment F). Participants were randomized to 1 of 3 sequences, in a ratio of 1:1:1. All participants received Treatments D, E, and F according to the treatment sequence which the participant was randomized to: DEF, EFD, or FDE. PK blood samples were collected for danicopan predose and for 72 hours following each danicopan administration.

### Both Parts

In both Parts 1 and 2 of the study, there was a washout period of at least 5 days between each danicopan dosing.

### Discussion of Study Design

Participants were randomized to treatment sequences to minimize assignment bias. A 3 period, 3 sequence crossover design was used to reduce the residual variability as every participant acted as their own control.

In each period, participants were confined to the clinic from Day-1 of Period 1, at the time indicated by the study center, and until after the 72-hour PK sample collection and study procedures on Day 4 of Period 3. Participants returned for study follow-up.

At all times, a participant may have been required to remain at the study center for longer at the discretion of the Investigator or designee.

All participants who received at least one dose of study drug (including participants who terminated the study early) returned to the study center 10 (+2) days after the last study drug administration for follow-up procedures, and to determine if any adverse events (AE) had occurred since the last study visit.

### Pharmacokinetics

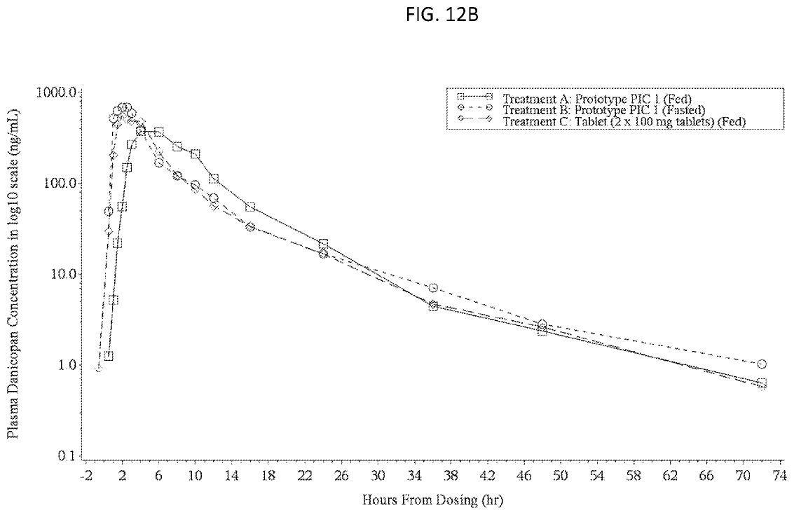

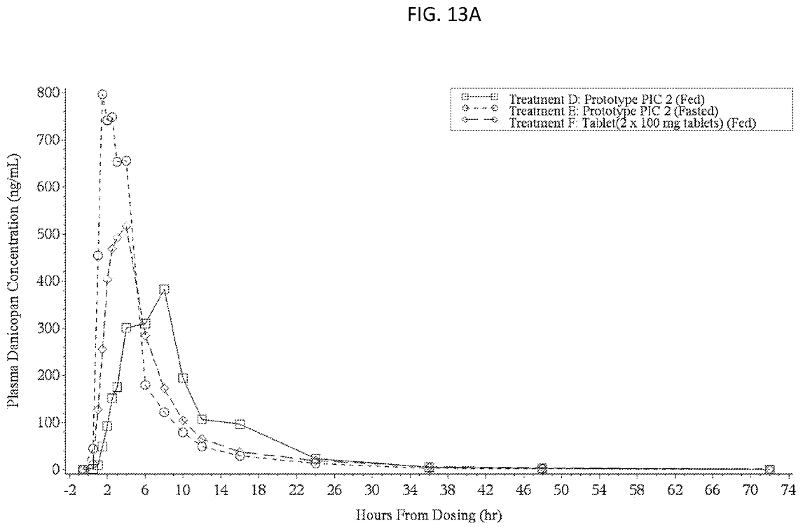

The mean plasma danicopan concentration-time profiles following single-dose 200 mg danicopan administered as a prototype PIC 1 formulation under fed and fasted conditions and as a tablet formulation under fed conditions are presented on a linear scale in FIG. 12A and on a semi-log scale in FIG. 12B.

Following a single oral dose of 200 mg danicopan, concentrations of danicopan were generally quantifiable in plasma by 0.5 hour (the first postdose time point) in all participants for each treatment. Danicopan was readily absorbed following administration of the PIC 1 formulation under fasted conditions (Treatment B) and tablet formulation under fed conditions (Treatment C), with maximum individual plasma concentrations occurring between 1.00 and 4.00 hours postdose. In comparison, a delay in absorption was observed when the PIC 1 formulation was administered under fed conditions (Treatment A), and maximum individual plasma concentrations were observed between 3.00 and 10.00 hours postdose. Concentrations after the peak declined in a multiphasic manner and remained quantifiable until 72 hours postdose for the majority of participants in each treatment. The peak mean concentration was lower for the PIC 1 formulation under fed conditions compared to fasted conditions as well as the tablet formulation under fed conditions.

A summary of plasma danicopan PK parameter estimates following each treatment is presented in Table 12. The statistical comparisons of plasma danicopan PK parameter estimates are summarized in Table 13 and in Table 14.

TABLE 12
Summary of Plasma Danicopan Pharmacokinetics Following Administration of 200 mg Danicopan
as Prototype PIC 1 Fed (Treatment A) and Fasted (Treatment B) and as Tablet Fed (Treatment
C) to Healthy Adult Subjects - Part 1 (Pharmacokinetic Population).

| Pharmacokinetic | | | |
|---|---|---|---|
| Parameters | Prototype PIC 1 Fed | Prototype PIC 1 Fasted | Tablet Fed |
| AUC₀₋ₜ (ng*hr/mL) | 3454 (25.1) [n = 12] | 3649 (32.7) [n = 12] | 3417 (22.6) [n = 12] |
| AUC₀₋ᵢₙₜ (ng*hr/mL) | 3465 (25.1) [n = 12] | 3663 (32.8) [n = 12] | 3428 (22.6) [n = 12] |
| AUC % extrap (%) | 0.1652 (154.5) [n = 12] | 0.2046 (185.8) [n = 12] | 0.1674 (189.0) [n = 12] |
| Cₘₐₓ (ng/mL) | 519.5 (28.9) [n = 12] | 871.8 (39.8) [n = 12] | 688.2 (33.1) [n = 12] |
| Tₘₐₓ (hr) | 6.009 (3.00, 10.00) [n = 12] | 2.001 (1.00, 4.00) [n = 12] | 2.997 (1.50, 4.00) [n = 12] |
| Kₑₗ (1/hr) | 0.07943 ± 0.028145 [n = 12] | 0.08769 ± 0.037354 [n = 12] | 0.07950 ± 0.027379 [n = 12] |
| t_{1/2} (hr) | 9.855 ± 3.7051 [n = 12] | 9.375 ± 3.8853 [n = 12] | 10.108 ± 4.7450 [n = 12] |
| CL/F (L/hr) | 59.44 ± 15.795 [n = 12] | 57.35 ± 19.974 [n = 12] | 59.76 ± 14.553 [n = 12] |
| Vz/F (L) | 808.9 ± 272.79 [n = 12] | 765.4 ± 380.35 [n = 12] | 843.4 ± 354.87 [n = 12] |

Prototype PIC 1 Fed: Danicopan (200 mg, made up of 1 × 200 mg prototype PIC 1) administered orally on Day 1, 30 minutes after the start of a moderate-fat meal
Prototype PIC 1 Fasted: Danicopan (200 mg, made up of 1 × 200 mg prototype PIC 1) administered orally on Day 1, under fasting conditions
Tablet Fed: Danicopan (200 mg, made up of 2 × 100 mg tablets) administered orally on Day 1, 30 minutes after the start of a moderate-fat meal
AUCs and Cₘₐₓ are presented as geometric mean (geometric CV %).
Tₘₐₓ is presented as median (minimum, maximum).
Other parameters are presented as arithmetic mean ± SD.

TABLE 13
Summary of Statistical Comparisons of Cₘₐₓ, AUC₀₋ₜ, and AUC_{0-inf} Following 200 mg
Danicopan Administered as Prototype PIC 1 Fed (Treatment A) and Fasted (Treatment B) and
as Tablet Fed (Treatment C) to Healthy Adult Subjects-Part 1 (Pharmacokinetic Population)

| | | Test | | Reference | | | | |
|---|---|---|---|---|---|---|---|---|
| | | Geometric | | Geometric | | | 90% Confidence | Intra-subject |
| Comparison | Parameter | LSMs | n | LSMs | n | GMR (%) | Interval | CV % |
| Treatment A vs | AUC₀₋ₜ (ng*hr/mL) | 3454 | 12 | 3417 | 12 | 101.10 | 92.67-110.30 | 12.42 |
| C | AUC_{0-inf} (ng*hr/mL) | 3465 | 12 | 3428 | 12 | 101.08 | 92.59-110.33 | 12.50 |
| | Cₘₐₓ (ng/mL) | 519.5 | 12 | 688.2 | 12 | 75.48 | 62.51-91.15 | 27.27 |
| Treatment A vs | AUC₀₋ₜ (ng*hr/mL) | 3454 | 12 | 3649 | 12 | 94.67 | 86.77-103.28 | 12.42 |
| B | AUC_{0-inf} (ng*hr/mL) | 3465 | 12 | 3663 | 12 | 94.61 | 86.67-103.28 | 12.50 |
| | Cₘₐₓ (ng/mL) | 519.5 | 12 | 871.8 | 12 | 59.59 | 49.35-71.95 | 27.27 |
| Treatment B vs | AUC₀₋ₜ (ng*hr/mL) | 3649 | 12 | 3417 | 12 | 106.80 | 97.89-116.51 | 12.42 |
| C | AUC_{0-inf} (ng*hr/mL) | 3663 | 12 | 3428 | 12 | 106.83 | 97.87-116.62 | 12.50 |
| | Cₘₐₓ (ng/mL) | 871.8 | 12 | 688.2 | 12 | 126.68 | 104.91-152.96 | 27.27 |

Treatment A: Danicopan (200 mg, made up of 1 × 200 mg prototype PIC 1) administered orally on Day 1, 30 minutes after the start of a moderate-fat meal
Treatment B: Danicopan (200 mg, made up of 1 × 200 mg prototype PIC 1) administered orally on Day 1, under fasting conditions
Treatment C: Danicopan (200 mg, made up of 2 × 100 mg tablets) administered orally on Day 1, 30 minutes after the start of a moderate-fat meal
Parameters were In-transformed prior to analysis
Test is the first treatment in the comparison and reference is the second.
Geometric least-squares means (LSMs) are calculated by exponentiating the LSMs from the ANOVA.
Geometric Mean Ratio (GMR) = 100*(test/reference)
Intra-subject CV % was calculated as 100 × square root(exp[MSE]-1), where MSE = Residual variance from ANOVA.

TABLE 14
Nonparametric Statistical Comparison of Tₘₐₓ Following 200 mg Danicopan
Administered as Prototype PIC 1 Fed (Treatment A) and Fasted (Treatment B) and as Tablet Fed (Treatment C)
to Healthy Adult Subjects-Part 1 (Pharmacokinetic Population)

| | | | | Difference Test-Reference | | |
|---|---|---|---|---|---|---|
| | | | | | 90% | |
| | | Median | | Median | Confidence | |
| Parameter | Comparison | Test | Reference | Difference | Interval | p-value |
| Tₘₐₓ | Treatment A vs C | 6.009 | 2.997 | 3.02 | (2.03, 4.02) | 0.0010 |
| | Treatment A vs B | 6.009 | 2.001 | 4.00 | (3.00, 5.00) | 0.0010 |
| | Treatment B vs C | 2.001 | 2.997 | −0.99 | (−1.50, −0.25) | 0.0342 |

Treatment A: Danicopan (200 mg, made up of 1 × 200 mg prototype PIC 1) administered orally on Day 1, 30 minutes after the start of a moderate-fat meal
Treatment B: Danicopan (200 mg, made up of 1 × 200 mg prototype PIC 1) administered orally on Day 1, under fasting conditions
Treatment C: Danicopan (200 mg, made up of 2 × 100 mg tablets) administered orally on Day 1, 30 minutes after the start of a moderate-fat meal
Test is the first treatment in the comparison and reference is the second.
p-value is from Wilcoxon Signed Rank Test.
The 90% confidence interval is constructed using Walsh Averages and appropriate quantile of the Wilcoxon Signed Ranks Test Statistic.

Following a single oral dose of 200 mg danicopan, the median Tₘₐₓ of danicopan in plasma was delayed for the PIC 1 formulation under fed conditions compared to fasted conditions as well as the tablet formulation under fed conditions. The terminal elimination phase was readily characterized for all participants for each treatment, and the apparent terminal half-life (t_{1/2}) estimates for danicopan were comparable for each treatment, ranging from approximately 9.38 to 10.1 hours. The apparent total clearance (CL/F) and apparent volume of distribution (V_{z}/F) estimates were also comparable for each treatment, ranging from 57.4 to 59.8 L/hr and 765 to 843 L, respectively.

Inter-participant variability (geometric CV %) for the exposure parameters (AUCs and Cₘₐₓ) of danicopan in plasma was generally low for each treatment, ranging from 25.1 to 28.9%, 32.7 to 39.8%, and 22.6% to 33.1% for PIC 1 (fed), PIC 1 (fasted), and tablet (fed), respectively.

Under fed conditions the extent of danicopan exposure in plasma was similar between the PIC 1 formulation and the tablet formulation, as the geometric mean ratios (GMRs; Treatment A/C) for AUC₀₋ₜ and AUC_{0-inf} were approximately 100%. In contrast, the peak exposure (Cₘₐₓ) to danicopan was approximately 25% lower for PIC 1 (fed) compared to tablet (fed) and the lower bound of the 90% confidence interval (CI) for the Cₘₐₓ GMR was below 80% (90% CI: 62.51-91.15). A statistically significant (p=0.0010) approximately 3-hour delay in median Tₘₐₓ was observed for danicopan for the PIC 1 formulation compared to the tablet formulation under fed conditions.

Following PIC 1 administration, the extent of danicopan exposure in plasma (as measured by AUC₀₋ₜ and AUC_{0-inf}) was similar under fed and fasted conditions, whereas peak exposure was approximately 40% lower in the presence of food. The GMR (Treatment A/B) for Cₘₐₓ was 59.59 and the 90% CI was entirely below 80% (49.35-71.95). A statistically significant (p=0.0010) approximately 4-hour delay in median Tₘₐₓ was observed for the PIC 1 formulation when given with food compared to fasted conditions.

The extent of danicopan exposure (AUC) was similar and the peak exposure (Cₘₐₓ) was approximately 27% higher for the PIC 1 formulation under fasted conditions compared to the tablet formulation under fed conditions. The median Tₘₐₓ occurred approximately 1 hour earlier for the PIC 1 formulation (fasted) compared to the tablet formulation (fed) (p=0.0342).

Of note, one participant (Participant 4) in Part 1 had a measurable predose concentration in Treatment C (tablet, fed condition). As the concentration value was ≤5% of Cₘₐₓ, the PK data for this participant were included in the PK summaries and statistical analyses.

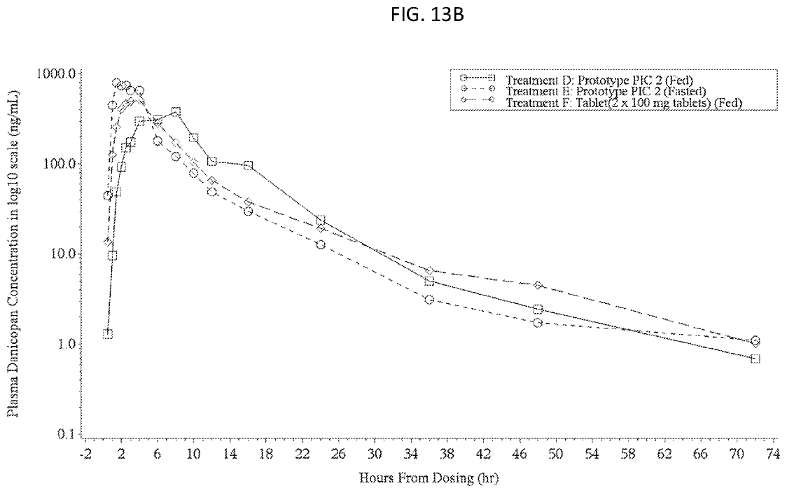

Mean plasma danicopan concentration-time profiles following single-dose 200 mg danicopan administered as a prototype PIC 2 formulation under fed and fasted conditions and as a tablet formulation under fed conditions are presented on a linear scale in FIG. 13A and on a semi-log scale in FIG. 13B.

Following a single oral dose of 200 mg danicopan, concentrations of danicopan were quantifiable in plasma by 0.5 hour postdose in all participants for each treatment. Danicopan was readily absorbed following administration of the PIC 2 formulation under fasted conditions (Treatment E) and the tablet formulation under fed conditions (Treatment F), with maximum individual plasma concentrations occurring between 1.50 and 6.00 hours postdose. In comparison, a delay in absorption was observed when the PIC 2 formulation was administered under fed conditions (Treatment D), and maximum individual plasma concentrations were observed between 4.01 and 16.00 hours postdose. Concentrations after the peak declined in a multiphasic manner and remained quantifiable until 72 hours postdose for the majority of participants in each treatment. The peak mean concentration was lower for the PIC 2 formulation under fed conditions compared to fasted conditions as well as the tablet formulation under fed conditions.

A summary of plasma danicopan PK parameters following each treatment is presented in Table 15. The statistical comparisons of plasma danicopan PK parameter estimates are summarized in Table 16 and Table 17.

TABLE 15
Summary of Plasma Danicopan Pharmacokinetics Following Administration of 200 mg Danicopan
as Prototype PIC 2 Fed (Treatment D) and Fasted (Treatment E) and as Tablet Fed
(Treatment F) to Healthy Adult Subjects - Part 2 (Pharmacokinetic Population)

| Pharmacokinetic | | | |
|---|---|---|---|
| Parameters | Prototype PIC 2 Fed | Prototype PIC 2 Fasted | Tablet Fed |
| AUC₀₋ₜ (ng*hr/mL) | 3229 (75.7) [n = 12] | 4009 (28.8) [n = 12] | 3606 (23.3) [n = 12] |
| AUC_{0-inf} (ng*hr/mL) | 3241 (75.5) [n = 12] | 4020 (28.8) [n = 12] | 3624 (23.2) [n = 12] |
| AUC % extrap (%) | 0.2467 (126.0) [n = 12] | 0.1941 (137.9) [n = 12] | 0.2700 (163.4) [n = 12] |
| Cₘₐₓ (ng/mL) | 444.0 (81.0) [n = 12] | 1167 (36.0) [n = 12] | 665.1 (34.6) [n = 12] |
| Tₘₐₓ (hr) | 6.001 (4.01, 16.00) [n = 12] | 2.757 (1.50, 4.00) [n = 12] | 3.494 (2.00, 6.00) [n = 12] |
| Kₑₗ (1/hr) | 0.08222 ± 0.026168 [n = 12] | 0.08514 ± 0.030487 [n = 12] | 0.06967 ± 0.022044 [n = 12] |
| t_{1/2} (hr) | 9.557 ± 4.0732 [n = 12] | 9.337 ± 3.7856 [n = 12] | 10.808 ± 3.0938 [n = 12] |
| CL/F (L/hr) | 83.06 ± 98.300 [n = 12] | 51.60 ± 14.587 [n = 12] | 56.54 ± 13.107 [n = 12] |
| Vz/F (L) | 1007 ± 930.83 [n = 12] | 690.0 ± 329.31 [n = 12] | 882.5 ± 344.07 [n = 12] |

Prototype PIC 2 Fed: Danicopan (200 mg, made up of 1 × 200 mg prototype PIC 2) administered orally on Day 1, 30 minutes after the start of a moderate-fat meal
Prototype PIC 2 Fasted: Danicopan (200 mg, made up of 1 × 200 mg prototype PIC 2) administered orally on Day 1, under fasting conditions
Tablet Fed: Danicopan (200 mg, made up of 2 × 100 mg tablets) administered orally on Day 1, 30 minutes after the start of a moderate-fat meal
AUCs and Cmax are presented as geometric mean (geometric CV %).
Tₘₐₓ is presented as median (minimum, maximum).
Other parameters are presented as arithmetic mean ± SD.

TABLE 16
Summary of Statistical Comparisons of Cₘₐₓ, AUC₀₋ₜ, and AUC_{0-inf} Following 200 mg
Danicopan Administered as Prototype PIC 2 Fed (Treatment D) and Fasted (Treatment E) and
as Tablet Fed (Treatment F) to Healthy Adult Subjects-Part 2 (Pharmacokinetic Population)

| | | Test | | Reference | | | | |
|---|---|---|---|---|---|---|---|---|
| | | Geometric | | Geometric | | | 90% Confidence | Intra-subject |
| Comparison | Parameter | LSMs | n | LSMs | n | GMR (%) | Interval | CV % |
| Treatment D vs | AUC₀₋ₜ (ng*hr/mL) | 3229 | 12 | 3606 | 12 | 89.55 | 69.95-114.64 | 36.19 |
| F | AUC_{0-inf} (ng*hr/mL) | 3241 | 12 | 3624 | 12 | 89.42 | 69.87-114.45 | 36.15 |
| | Cₘₐₓ (ng/mL) | 444.0 | 12 | 665.1 | 12 | 66.77 | 49.57-89.92 | 44.25 |
| Treatment D vs | AUC₀₋ₜ (ng*hr/mL) | 3229 | 12 | 4009 | 12 | 80.56 | 62.93-103.13 | 36.19 |
| E | AUC_{0-inf} (ng*hr/mL) | 3241 | 12 | 4020 | 12 | 80.60 | 62.98-103.16 | 36.15 |
| | Cₘₐₓ (ng/mL) | 444.0 | 12 | 1167 | 12 | 38.06 | 28.25-51.26 | 44.25 |
| Treatment E vs F | AUC₀₋ₜ (ng*hr/mL) | 4009 | 12 | 3606 | 12 | 111.16 | 86.83-142.31 | 36.19 |
| | AUC_{0-inf} (ng*hr/mL) | 4020 | 12 | 3624 | 12 | 110.94 | 86.68-141.99 | 36.15 |
| | Cₘₐₓ (ng/mL) | 1167 | 12 | 665.1 | 12 | 175.44 | 130.26-236.30 | 44.25 |

Treatment D: Danicopan (200 mg, made up of 1 × 200 mg prototype PIC 2) administered orally on Day 1, 30 minutes after the start of a moderate-fat meal
Treatment E: Danicopan (200 mg, made up of 1 × 200 mg prototype PIC 2) administered orally on Day 1, under fasting conditions
Treatment F: Danicopan (200 mg, made up of 2 × 100 mg tablets) administered orally on Day 1, 30 minutes after the start of a moderate-fat meal
Parameters were In-transformed prior to analysis
Test is the first treatment in the comparison and reference is the second.
Geometric least-squares means (LSMs) are calculated by exponentiating the LSMs from the ANOVA.
Geometric Mean Ratio (GMR) = 100*(test/reference)
Intra-subject CV % was calculated as 100 × square root(exp[MSE]-1), where MSE = Residual variance from ANOVA.

TABLE 17
Nonparametric Statistical Comparison of Tmax Following 200 mg Danicopan Administered as Prototype
PIC 2 Fed (Treatment D) and Fasted (Treatment E) and as Tablet Fed (Treatment F)
to Healthy Adult Subjects-Part 2 (Pharmacokinetic Population)

| | | | | Difference Test-Reference | | |
|---|---|---|---|---|---|---|
| | | | | | 90% | |
| | | Median | | Median | Confidence | p- |
| Parameter | Comparison | Test | Reference | Difference | Interval | value |
| Tₘₐₓ | Treatment D vs F | 6.001 | 3.494 | 3.50 | (2.50, 4.49) | 0.0010 |
| | Treatment D vs E | 6.001 | 2.757 | 4.25 | (3.00, 6.00) | 0.0005 |
| | Treatment E vs F | 2.757 | 3.494 | −0.99 | (−2.00, 0.01) | 0.2334 |

Treatment D: Danicopan (200 mg, made up of 1 × 200 mg prototype PIC 2) administered orally on Day 1, 30 minutes after the start of a moderate-fat meal
Treatment E: Danicopan (200 mg, made up of 1 × 200 mg prototype PIC 2) administered orally on Day 1, under fasting conditions
Treatment F: Danicopan (200 mg, made up of 2 × 100 mg tablets) administered orally on Day 1, 30 minutes after the start of a moderate-fat meal
Test is the first treatment in the comparison and reference is the second.
p-value is from Wilcoxon Signed Rank Test.
The 90% confidence interval is constructed using Walsh Averages and appropriate quantile of the Wilcoxon Signed Ranks Test Statistic.

Following a single oral dose of 200 mg danicopan, the median Tₘₐₓ of danicopan in plasma was delayed for the PIC 2 formulation under fed conditions compared to fasted conditions as well as the tablet formulation under fed conditions. The terminal elimination phase was readily characterized for all participants for each treatment, and the apparent terminal ty, estimates for danicopan were comparable following each treatment, ranging from approximately 9.34 to 10.8 hours. The apparent CL/F and V_{z}/F estimates for danicopan were higher for the PIC 2 formulation under fed conditions compared to fasted conditions as well as the tablet formulation under fed conditions.

Inter-participant variability (geometric CV %) for the exposure parameters (AUCs and Cₘₐₓ) of danicopan was generally low for the PIC 2 formulation under fasted conditions and tablet formulation under fed conditions (range: 28.8 to 36.0% and 23.2 to 34.6%, respectively). Moderate inter-participant variability was observed for the PIC 2 formulation under fed conditions, ranging from 75.5 to 81.0%.

The GMRs (Treatment D/F) for the AUC₀₋ₜ, AUC_{0-inf}, and Cₘₐₓ of danicopan in plasma showed a decrease in exposure of approximately 10%, 11%, and 33%, respectively, for the PIC 2 formulation compared to the tablet formulation under fed conditions. The 90% CI for the GMRs of AUC₀₋ₜ and AUC_{0-inf} contained 100% (69.95-114.64 and 69.87-114.45, respectively), in contrast to the 90% CI for the GMR of Cₘₐₓ (49.57-89.92). However, the lower 90% CI bound was below 80% for each parameter. A statistically significant (p=0.0010) approximately 3.5-hour delay in median Tₘₐₓ was observed for danicopan for the PIC 2 formulation in comparison to the tablet formulation under fed conditions.

The GMRs (Treatment D/E) for AUC and Cₘₐₓ of danicopan showed a decrease of approximately 19% and 60%, respectively, when the PIC 2 formulation was administered under fed conditions compared to fasted conditions. The 90% CI for the GMRs of AUC₀₋ₜ and AUC_{0-inf} contained 100% (62.93-103.13 and 62.98-103.16, respectively), though the lower bound was below 80%. The 90% CI for the GMR of Cₘₐₓ was entirely below 80% (28.25-51.26). A statistically significant (p=0.0005) approximately 4.25-hour delay in median Tₘₐₓ was observed for the PIC 2 formulation when given with food compared to fasted conditions.

The AUC and Cₘₐₓ of danicopan were approximately 11% and 75% higher, respectively, for the PIC 2 formulation under fasted conditions compared to the tablet formulation under fed conditions while the median Tₘₐₓ estimates for each treatment were not statistically different.

Pharmacokinetic Conclusions from Part 1 of Study

Following a single 200-mg dose of danicopan, the extent of exposure (AUC) to danicopan for the prototype PIC 1 and tablet formulations were comparable under fed (moderate-fat meal) conditions. However, peak exposure (Cₘₐₓ) to danicopan was approximately 25% lower for the PIC 1 formulation. An approximate 3-hour delay in the median Tₘₐₓ of danicopan was observed for the PIC 1 formulation compared to the tablet formulation under fed conditions.

Following a single 200-mg dose of danicopan in the prototype PIC 1 formulation, the extent of danicopan exposure was comparable with food as compared to fasted conditions. However, peak danicopan exposure was approximately 40% lower under fed conditions. An approximate 4-hour delay in the median Tₘₐₓ of danicopan was observed under fed conditions compared to fasted conditions.

The PIC 1 formulation demonstrates a decrease in the rate of absorption (lower Cₘₐₓ) with a similar systemic exposure to danicopan compared to the tablet formulation under fed conditions.

The food effect on the PIC 1 formulation (lower Cₘₐₓ) contrasts with previously observed increases in danicopan systemic exposure when the tablet formulation is administered with food.

The extent of exposure to danicopan was comparable following administration of PIC 1 under fasted conditions and tablet under fed conditions. However, peak exposure to danicopan was 27% higher and the median Tₘₐₓ was 1 hour earlier for PIC 1 (fasted) compared to the tablet (fed).

Pharmacokinetic Conclusions from Part 2 of Study

Following a single 200 mg dose of danicopan, the extent of exposure was slightly lower (10-11%) for the prototype PIC 2 formulation compared to the tablet formulation under fed conditions. Peak exposure to danicopan was approximately 33% lower and the median Tₘₐₓ was delayed by approximately 3.5 hours for the PIC 2 formulation compared to tablet.

Administration of a single 200 mg dose of danicopan in the prototype PIC 2 formulation with food reduced both the extent of exposure and peak exposure to danicopan by approximately 19% and 62%, respectively, compared to fasted conditions. An approximate 4.25 hour delay in the median Tₘₐₓ of danicopan was observed under fed conditions compared to fasted conditions.

The PIC 2 formulation demonstrates a reduction in both the overall systemic exposure to danicopan and a decrease in the rate of absorption (lower Cₘₐₓ) compared to the tablet formulation under fed conditions.

The food effect on the PIC 2 formulation (lower AUC and Cₘₐₓ) contrasts with previously observed increases in danicopan systemic exposure when the tablet formulation is administered with food

Overall, a more pronounced food effect on danicopan exposure is observed for the PIC 2 formulation compared to the PIC 1 formulation.

The extent of exposure to danicopan was slightly increased (11%) following administration of PIC 2 under fasted conditions compared to the tablet formulation under fed conditions. Peak exposure to danicopan was approximately 75% higher for PIC 2 (fasted). The median Tₘₐₓ for danicopan was comparable for both treatments.

### OTHER EMBODIMENTS

While the present disclosure has been described in connection with specific embodiments thereof, it will be understood that it is capable of further modifications and this application is intended to cover any variations, uses, or adaptations of the present disclosure following, in general, the principles of the present disclosure and including such departures from the present disclosure come within known or customary practice within the art to which the present disclosure pertains and may be applied to the essential features hereinbefore set forth.

All publications, patents and patent applications are herein incorporated by reference in their entirety to the same extent as if each individual publication, patent or patent application was specifically and individually indicated to be incorporated by reference in its entirety.

We claim:

1. A solid dispersion comprising: (i) substantially amorphous danicopan; and (ii) a pharmaceutically acceptable polymer, wherein the (w/w) ratio of substantially amorphous danicopan to pharmaceutically acceptable polymer in the solid dispersion is from 10:1 to 1:4.

2. The solid dispersion of claim 1, wherein the percentage loading of substantially amorphous danicopan is from 25% to 90% (w/w).

3. The solid dispersion of claim 2, wherein the (w/w) ratio of substantially amorphous danicopan to the pharmaceutically acceptable polymer is from 3:1 to 4:1.

4. The solid dispersion of any one of claims 1-3, wherein by X-ray powder diffraction at least 90% of danicopan is in amorphous form.

5. The solid dispersion of any one of claims 1-4, wherein the pharmaceutically acceptable polymer comprises a polymer selected from the group consisting of a cellulose derivative, a polyacrylate, a polyvinyl pyrrolidone, a polyvinyl acetate, a copolymer of a polyvinyl pyrrolidone and a polyvinyl acetate, and combinations thereof.

6. The solid dispersion of claim 5, wherein the pharmaceutically acceptable polymer is a cellulose derivative selected from the group consisting of a cellulose acetate having from 10% to 50% acetyl, an alkyl cellulose, a hydroxyalkyl cellulose, a hydroxyalkylalkyl cellulose, a hydroxyalkylalkyl cellulose ester, a carboxyalkyl cellulose, a carboxyalkylalkyl cellulose, and a carboxyalkyl cellulose ester.

7. The solid dispersion of claim 6, wherein the pharmaceutically acceptable polymer is hydroxypropyl methylcellulose (HPMC) E3.

8. The solid dispersion of claim 6, wherein the pharmaceutically acceptable polymer is a cellulose acetate selected from the group consisting of cellulose acetate phthalate (CAP), methylcellulose acetate phthalate, hydroxypropylmethyl cellulose (HPMC), and hydroxypropylmethyl cellulose acetate succinate (HPMCAS).

9. The solid dispersion of claim 8, wherein the pharmaceutically acceptable polymer is a HPMCAS polymer selected from grade L (HPMCAS-L), grade H (HPMCAS-H), or grade M (HPMCAS-M).

10. The solid dispersion of claim 5, wherein the pharmaceutically acceptable polymer is a polyacrylate selected from the group consisting of a polymethacrylate, a methacrylate copolymer, and an ethacrylate copolymer.

11. The solid dispersion of claim 10, wherein the pharmaceutically acceptable polymer is Eudragit L-100.

12. The solid dispersion of claim 5, wherein the pharmaceutically acceptable polymer is a polyvinyl acetate selected from the group consisting of a polyvinyl acetate ester and a polyethylene glycol-polyvinylcaprolactam-polyvinylacetate copolymer.

13. The solid dispersion of claim 12, wherein the pharmaceutically acceptable polymer is polyvinylacetate phthalate (PVAP).

14. The solid dispersion of claim 5, wherein the pharmaceutically acceptable polymer is a copolymer of a polyvinyl pyrrolidone and a polyvinyl acetate and said copolymer has from 10:90 to 70:30 ratio of N-vinyl-2-pyrrolidone to vinyl acetate.

15. A pharmaceutical composition in a capsule or tablet for oral administration comprising a solid dispersion of any one of claims 1-14.

16. The pharmaceutical composition of claim 15, containing from 20 mg to 900 mg of substantially amorphous danicopan.

17. The pharmaceutical composition of claim 15 or 16, further comprising a pharmaceutically acceptable plasticizer, binder, filler, carrier, excipient, and/or surfactant.

18. The pharmaceutical composition of any one of claims 15-17, wherein the solid dispersion is formed by spray drying a liquid mixture comprising danicopan and the pharmaceutically acceptable polymer.

19. The pharmaceutical composition of any one of claims 15-18, wherein the pharmaceutical composition is in the form of a capsule selected from a hard hydroxy propyl methylcellulose capsule, hard gelatin capsule, or soft gelatin capsule, wherein the capsule comprises a powder comprising the solid dispersion.

20. The pharmaceutical composition of any one of claims 15-18, wherein the pharmaceutical composition is in the form of a tablet.

21. The pharmaceutical composition of any one of claims 15-20, when tested in accordance with the dissolution method defined herein employing 0.1 N hydrochloric acid in water at 37° C. with 50 rpm stirring, dissolves at least 50% (w/w) of the pharmaceutical composition within the first 8-90 minutes of the test.

22. The pharmaceutical composition of any one of claims 15-20, when tested in accordance with the supersaturation profiling method defined herein employing simulated gastric fluid (SGF) or simulated intestinal fluid (SIF) at 37° C., releases 0.027 or 0.8 mg/ml of the pharmaceutical composition in SIF or SGF, respectively, within the first four hours of the test.

23. A pharmaceutical composition for oral administration comprising danicopan dissolved in a glyceride fatty acid ester, a pharmaceutically acceptable organic solvent, and a D-α-tocopherol polyethylene glycol succinate (TPGS) compound contained in a liquid soft gelatin capsule.

24. The pharmaceutical composition of claim 23, wherein the ratio (w/w) of glyceride fatty acid ester, danicopan, pharmaceutically acceptable organic solvent, and TPGS is 16:2:1:1.

25. The pharmaceutical composition of claim 24, wherein the danicopan is dissolved in Capmul MCM, propylene glycol, and D-alpha-tocopheryl PEG 1000 succinate.

26. The pharmaceutical composition of any one of claims 23-25, comprising from 20 mg to 900 mg of danicopan.

27. A method of preparing a solid dispersion of any one of claims 1-14, said method comprising:
(i) dissolving danicopan and a pharmaceutically acceptable polymer in an organic solvent to form a solution; and
(ii) spray drying said solution to form the solid dispersion.

28. The method of claim 27, wherein the organic solvent has a boiling point of less than 80° C.

29. A method of treating a condition modulated by complement factor D comprising orally administering to a subject in need thereof an effective amount of the pharmaceutical composition of any one of claims 15-26.

30. The method of claim 29, wherein the pharmaceutical composition is orally administered once, twice, or three times per day.

31. The method of claim 29 or 30, wherein the condition is paroxysmal nocturnal hemoglobinuria (PNH) and PNH with clinically evident extravascular hemolysis (EVH).

32. The method of claim 31, wherein the pharmaceutical composition comprises 150-200 mg of danicopan and is orally administered three times daily.

33. The method of claim 32, wherein the pharmaceutical composition comprises 150 mg of danicopan and is orally administered three times daily.

34. The method of claim 29 or 30, wherein the condition is geographic atrophy (GA) secondary to age-related macular degeneration (AMD).

35. The method of claim 34, wherein the pharmaceutical composition comprises 100-200 mg of danicopan and is orally administered twice daily.

36. The method of claim 34, wherein the pharmaceutical composition comprises 200-400 mg of danicopan and is orally administered once daily.
